(19) United States
(12) Patent Application Publication
(10) Pub. No.: US 2012/0244071 A1
(43) Pub. Date: Sep. 27, 2012

(54) FTY720 HALOGENATED DERIVATIVES

(75) Inventors: Yves Auberson, Allschwil (CH); Emmanuelle Briard, St. Louis (FR); David Orain, Hesingue (FR)

(73) Assignee: Novartis AG, Basel (CH)

(21) Appl. No.: 13/513,708

(22) PCT Filed: Dec. 8, 2010
(86) PCT No.: PCT/EP10/69169

§ 371 Date: Jun. 4, 2012

(30) Foreign Application Priority Data
Dec. 10, 2009 (EP) 09178664.0

(51) Int. Cl. A61K 51/04; A61B 6/00; A61B 6/03; C07C 217/64; C07F 9/22

(52) U.S. Cl. 424/1.85; 564/360; 562/11; 600/436

(57) ABSTRACT

There are provided new iodo- and bromo-compounds, and their use as diagnostic agents and imaging agents for diseases and disorders wherein S1P receptor expression is altered.

### NEW COMPOUNDS

The present invention relates to novel compounds in particular novel radioactive compounds, their preparation, and the use of such novel radioactive compounds as radiotracers/markers for imaging techniques and diagnostics tools in the field of diseases or disorders related to S1P receptors, such as autoimmune diseases, neurodegenerative diseases, brain diseases or demyelinating diseases, for example multiple sclerosis.

Multiple sclerosis (MS) is the chief cause of neurological disability in young adults and the most common demyelinating disorder of the central nervous system. MS takes several forms and almost any neurological symptoms can appear: The symptoms occur either in discrete attacks (relapsing forms) or slowly accumulating over time (progressive forms). Between the attacks, the symptoms may disappear, but often permanent neurological disorders occur, especially as the disease advances. As the signs and symptoms of MS may be similar to many other medical problems, that disease is difficult to diagnose. Diagnostic criteria have been established to facilitate and standardize the diagnostic process, including neuroimaging analysis and magnetic resonance imaging (MRI) of the brain and spine to visualize and follow the areas of demyelination (MS lesions or plaques).

But currently there is no diagnostic test which is perfectly specific to MS, only biopsies or post-mortem examinations can yield an absolutely certain diagnosis. Therefore, there is a strong medical need for an effective method to diagnose multiple sclerosis.

Non-invasive, nuclear imaging techniques can be used to obtain information on the physiology and biochemistry of living subjects, including experimental animals, patients and volunteers. These techniques rely on the use of imaging instruments that can detect radiation emitted from radiotracers administered to living subjects. The information obtained can be reconstructed to provide planar and tomographic images which reveal the distribution and/or concentration of the radiotracer as a function of time. Examples of such techniques which are particularly interesting for multiple sclerosis, brain diseases or demyelinating diseases, are Positron emission tomography (PET), a nuclear medicine imaging technique which produces a three-dimensional image or Single photon emission computed tomography (SPECT), a nuclear medicine tomographic imaging technique using gamma rays.

One of the requirements for the use of these techniques is the availability of an adequate tracer. Such a tracer may for example accumulates in specific organs or tissues; thus its visualization after administration permits to visualize these tissues or organs. Or it may have a characteristic activity (for example has a binding efficacy for specific receptors) which is distributed or anyhow modified in case of a disease or disorder (for example if such specific receptors are involved in these diseases or disorders); thus its visualization in the body will permit to detect, staging or follow-up such diseases or disorders. To be visualized that compound is radiolabelled. Therefore it is necessary that the radiolabeling does not alter the specific properties of the compound.

Many diseases or disorders are known or suspected to be anyhow related to the receptors of Sphingosine 1-phosphate (S1P). S1P is a bioactive sphingolipid that mediates diverse cellular responses such as proliferation, cytoskeletal organization and is involved in phenomenon such as regulation of immune cell trafficking, vascular homeostasis or cell communication in the central nervous system. S1P is contained in body fluids and tissues at different concentrations, and excessive production of the pleiotropic mediator at inflammatory sites may participate in various pathological conditions. Gene deletion studies and reverse pharmacology provided evidence that many effects of S1P are mediated via the five G-protein-coupled S1P receptor subtypes (S1P receptors). The receptors subtypes S1P1, S1P2 and S1P3 are widely expressed and represent the dominant receptors in the cardiovascular system. S1P1 is also a dominant receptor on lymphocytes and regulates their egress from secondary lymphatic organs. S1P4 receptors are expressed in the lymphoid system and S1P5 in the white matter tract of the central nervous system (CNS).

Interactions of synthetic ligands with these S1P receptors offer novel strategies for broad therapeutic applications.

The prototype S1P receptor modulator, FTY720 (fingolimod, 2-amino-2-[2-(4-octylphenyl)ethyl]propane-1,3-diol), targets four of the five S1P receptor subtypes and may act at several levels to modulate lymphocyte trafficking via lymphocytic and endothelial S1P1 and, perhaps, other inflammatory processes through additional S1P receptor subtypes. FTY720 binds with high-affinity to S1P1 (0.3 nM), S1P4 (0.6 nM) and S1P5 (0.3 nM) and with about 10-fold lower affinity to S1P3 (3.1 nM), but not to S1P2. Ongoing clinical trials indicate that FTY720 may provide an effective treatment of relapsing-remitting multiple sclerosis (as described in, for example, “FTY720 therapy exerts differential effects on T cell subsets in multiple sclerosis”, Mehling M et al., Neurology. 2008 Oct. 14; 71(16):1261-7).

There is also a need to prepare radiolabelled derivative of FTY720 that could be used to mimic the drug in order to further define the therapeutic action of FTY720, e.g. to quantify its pharmacokinetics and organ distribution in patients.

Surprisingly, the inventors have identified FTY720 derivatives which contain an atom which can be a radioactive isotope. Such derivatives are able to mimic FTY720 pharmacokinetics and physicochemical activities. For example, they can mimic one or more of the following FTY720 properties: organ distribution, affinity and selectivity for S1P receptors, phosphorylation kinetics.

### BRIEF DISCLOSURE OF THE INVENTION

In view of the properties of FTY720, there is a need to develop FTY720 derivatives which can be used as tracers, or imaging agents, i.e. which can mimic FTY720 properties despite the introduction of one or more radioisotopes.

Iodine and bromine are particularly heavy atoms (with respective atomic weights of ca. 127 and 80 Da), specially in comparison to FTY720 (molecular weight of 307.5). It is therefore surprising that despite the introduction of such halogen atoms, which are expected to modify the physicochemical and pharmacokinetic properties of the compounds, it is possible to prepare FTY720 derivatives which despite the introduction of the halogen atom can bind to S1P receptors with an affinity and selectivity profile close to FTY720, while maintaining similar pharmacokinetic properties. After radiolabeling, these compounds can be used for in vitro and in vivo imaging applications. When properly isotope-labeled, these agents exhibit valuable properties as histopathological labeling agents, imaging agents and/or biomarkers for the selective labeling of S1P receptors, e.g. for at least one of the subtypes S1P1, S1P3, S1P4 and S1P5. More particularly the compounds of the invention are useful as markers or radiotracers for labeling S1P receptors in vitro or in vivo, in particular for labeling at least one of the subtypes S1P1, S1P3, S1P4 and S1P5 receptors in vitro or in vivo.

Additionally, these compounds tend to accumulate in myelin, possibly by a mechanism that is independent of their affinity for S1P receptors, such as insertion into myelin sheets. They are hence also suitable to imaging myelin in diseases and disorders where the myelin sheet has been disturbed, e.g. in demyelinating diseases.

Suitable radionuclides that may be incorporated in the compounds of invention include: ¹²³I, ¹²⁴I, ¹²⁵I, ¹³¹I, ⁷⁵Br or ⁷⁶Br. The choice of the radionuclide to be incorporated into the compounds will depend on the specific analytical or pharmaceutical application. Therefore, for in vitro labeling of S1P receptors and for competition assays compounds that incorporate ¹²⁵I or ¹³¹I would be preferred. For diagnostic and investigative imaging agents (positron emission tomography (PET) or single photon emission computed tomography (SPECT)). In a specific embodiment of the invention, compounds that incorporate, respectively ¹²⁴I or ¹²³I are preferred.

These tracers can be used for imaging S1P receptors in tissue sections in vitro or in vivo, for example for analyzing the receptor occupancy of compounds having an affinity for the S1P receptors, and thus evaluate the potential therapeutic application of such compounds.

Such tracers may also be used for diagnosing, or staging diseases and disorders where S1P receptors expression is affected, for example autoimmune or demyelinating diseases, such as multiple sclerosis. They may also be used to evaluate the patient populations susceptible to benefit from treatment with a drug acting through interaction with SW receptors, or to estimate the distribution of FTY720 in specific patient populations.

### COMPOUNDS OF THE INVENTION

The present invention provides new derivatives of FTY720, in particular new radioactive derivatives of FTY720, i.e. radiolabeled derivatives of FTY720, the use of the radiolabelled derivatives of FTY720 as tracers for medical imaging in diagnostic and therapeutic applications.

As hereinabove defined, “derivatives of FTY720” refers to compounds having a structure identical or similar to FTY720 or FTY720-phosphate, and further containing at least one iodine or bromine atom, e.g. at least one radioactive isotope of iodine or bromine.

FTY720 is 2-amino-2-[2-(4-octylphenyl)ethyl]propane-1,3-diol, as shown

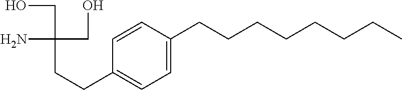

FTY720-phosphate refers to a phosphorylated form of FTY720, as shown

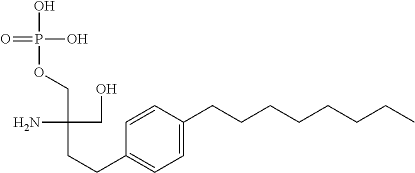

The terms “radiolabelled derivatives of FTY720” and “radiolabelled compounds of the invention” refer to the derivatives of FTY720 as described herein which are radioactive, i.e. wherein at least one iodine or bromine atom is substituted with, e.g. replaced by, an iodine or bromine radioactive isotope, for example, with a corresponding radioactive isotope. I.e. the radiolabelled compounds of the invention may contain at least one atom selected from ¹²³I, ¹²⁵I, ¹²⁴I, ¹³¹I, ⁷⁵Br and ⁷⁶Br, e.g. at least one atom selected from ¹²³I and ¹²⁴I.

The derivatives of FTY720 and radiolabelled derivatives of FTY720 according to the invention are compounds of formula I

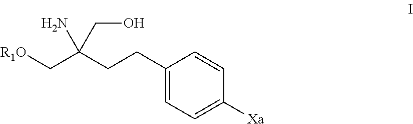

wherein

Xₐ is C₁₋₁₀ alkyl or OC₁₋₉alkyl, e.g. C₈ alkyl, e.g. n-octyl;

R₁ is H or C₁₋₆ alkyl, or PO₃H₂;

and wherein at least one hydrogen atom, e.g. at least one hydrogen atom linked to a carbon atom, is replaced by an iodine or bromine atom.

In the radiolabelled derivatives of FTY720 of Formula I, at least one hydrogen atom, e.g. at least one hydrogen atom linked to a carbon atom, is process with, e.g. replaced by, a radioactive isotope of iodine or bromine, e.g. with an atom selected from ¹²³I, ¹²⁵I, ¹²⁴I, ¹³¹I, ⁷⁵Br and ⁷⁶Br, e.g. with ¹²³I or ¹²⁴I.

In a specific embodiment, the radiolabelled derivatives of FTY720 of Formula I contains at least one atom selected from ¹²³I, ¹²⁵I, ¹²⁴I, ¹³¹I, ⁷⁵Br and ⁷⁶Br, e.g. at least one atom selected from ¹²³I and ¹²⁴I, e.g. contains ¹²³I or ¹²⁴I.

Preferably, the hydrogen atom which is substituted with, e.g. replaced by, a radioactive isotope is linked to a carbon atomradiolabelled

The iodine or bromine atom may be incorporated as a substituent on the aryl ring of the molecule, in which case the derivative is referred to as “iodine aryl FTY720 derivative” or “bromine aryl FTY720 derivative”. Such aryl FTY720 derivative may contain one or more iodine or bromine atom(s), e.g. at least one radioactive isotope of iodine or bromine.

The present invention provides a compound, e.g. a radiolabelled compound, of formula Ia

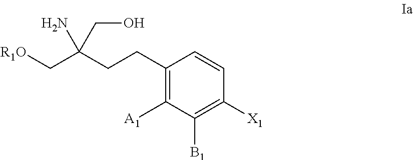

wherein
R₁ is as defined above;
at least one of A₁ and B₁ is I (iodine) or Br, the other being H; and
X₁ is C₁₋₁₀ alkyl or OC₁₋₉ alkyl, e.g. X₁ is C₈ alkyl

In one specific embodiment, R₁ is H or PO₃H₂.

In another embodiment, X₁ is n-octyl or n-heptyloxy.

For example, R₁ is H and X₁ is n-octyl, or R₁ is PO₃H₂ and X₁ is n-octyl.

In yet another embodiment, either A₁ is selected from the group consisting of I (iodine) and Br and B₁ is H, or A₁ is H and B₁ is selected from the group consisting of I (iodine) and Br.

Preferably,
R₁ is H or PO₃H₂;
at least one A₁ and B₁ is I (iodine), the other being H; and
X₁ is n-octyl or n-heptyloxy.

In the radiolabelled compounds of formula Ia, at least one iodine or bromine atom is substituted with, e.g. replaced by, an radioactive isotope of iodine or bromine, e.g. a radioactive isotope selected from ¹²⁵I, ¹²⁴I, ¹²³I, ¹³¹I, ⁷⁵Br and ⁷⁶Br, e.g. selected from ¹²⁵I and ¹²⁴I.

In another embodiment, the alkyl chain of the FTY720 derivative is terminated by a double bound wherein at least one of the carbon atom is substituted with, e.g. replaced by, one iodine or bromine, the derivative is then referred to as “iodine allyl FTY720 derivative” or “bromine allyl FTY720 derivative”.

The present invention further provides a compound, e.g. a radiolabelled compound, of formula Ib,

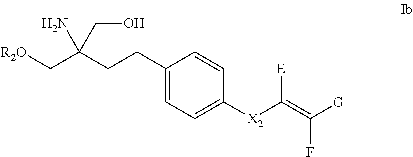

wherein
R₂ is H, C₁₋₆ alkyl, or PO₃H₂;
at least one of E, F and G is I (iodine) or Br, the others are H, for example at least one of F and G is selected from the group consisting of I (iodine) and Br, the others are H.
X₂ is C₁₋₈ alkyl or OC₁₋₇ alkyl.

In another embodiment, X₂ is 1,6-n-pentylene or oxy-n-butyl.

In one embodiment, R₂ is H or PO₃H₂.

In another embodiment either E, F or G is selected from the group consisting of I (iodine) and Br, the others being H; for example E and G are H and F is selected from the group consisting of I (iodine) and Br.

In yet a further embodiment, R₂ is PO₃H₂, at least one of E, F and G is I (iodine) or bromo, the others being H. For example R₂ is PO₃H₂, E is H and either G is I (iodine) or bromo and F is H or, reciprocally, G is H and F is I (iodine) or bromo.

In another embodiment E is selected from the group consisting of I (iodine) and bromo, and G and H are both H.

In the radiolabelled compounds of formula Ia, at least one iodine or bromine atom is substituted with, e.g. replaced by, an iodine or bromine radioactive isotope, e.g. a radioactive isotope selected from ¹²⁵I, ¹²⁴I, ¹²³I, ¹³¹I, ⁷⁵Br and ⁷⁶Br, e.g. selected from ¹²⁵I and ¹²⁴I.

In the definitions of the compounds of formula I, Ia and Ib as herein described, the terms iodine (“I”) and bromine (“Br”) refer, respectively, to iodine and bromine atoms, including all isotopes of such atoms.

Accordingly, the compounds of formula I, Ia and Ib, can be radiolabelled compounds, e.g. the iodine atom may be selected from ¹²⁵I, ¹²⁴I, ¹²³I, and ¹³¹I, and the bromo atom may be selected from ⁷⁵Br and ⁷⁶Br.

Preferably the compounds of the invention contain at least one radiolabelledradiolabelled atom, e.g. an atom selected from ¹²³I and ¹²⁴I.

When the compounds of formula I, Ia or Ib have one or more asymmetric centers in the molecule, the present invention is to be understood as embracing the various optical isomers, as well as racemates, diastereoisomers and mixtures thereof. Compounds of formula Ia or Ib, when the carbon atom bearing the amino group is asymmetric, have preferably the S-configuration at this carbon atom.

The compounds of formula I, Ia or Ib may exist in free or salt form. Examples of pharmaceutically acceptable salts of the compounds of the formula I, Ia or Ib include salts with inorganic acids, such as hydrochloride, hydrobromide and sulfate, salts with organic acids, such as acetate, fumarate, maleate, benzoate, citrate, malate, methanesulfonate and benzenesulfonate salts, or, when appropriate, salts with metals such as sodium, potassium, calcium and aluminium, salts with amines, such as triethylamine and salts with dibasic amino acids, such as lysine. The compounds and salts of the present invention encompass hydrate and solvate forms.

Examples of compounds of formula Ia, e.g. radiolabelled compounds of formula Ia, are

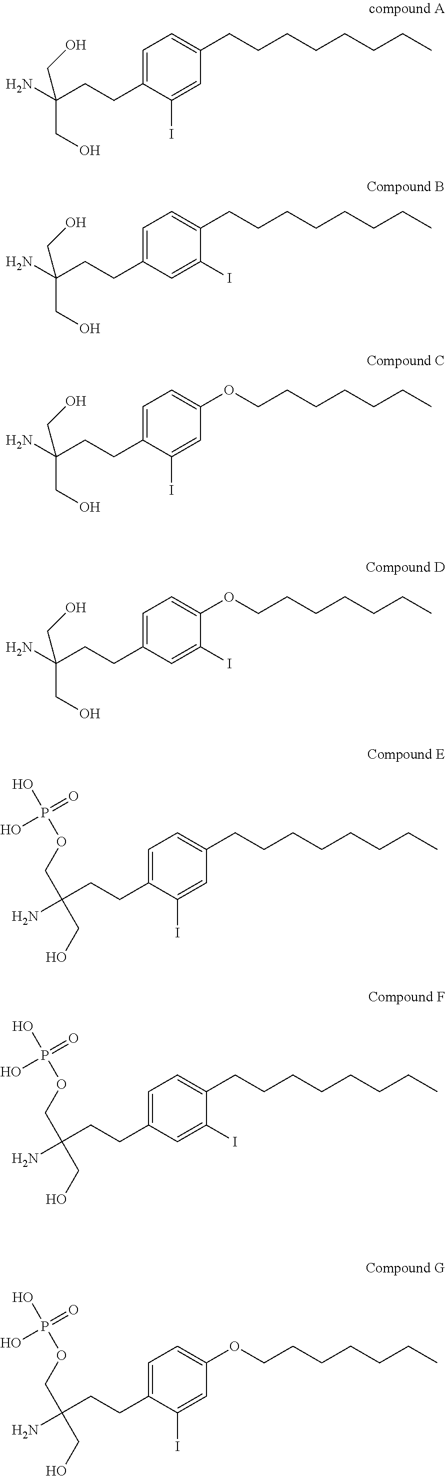

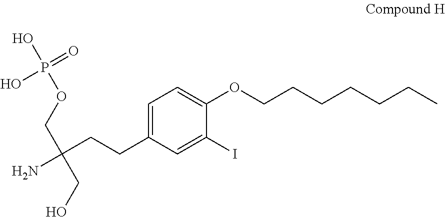

Examples of compound of formula Ib, e.g. radiolabelled compounds of formula Ia, are

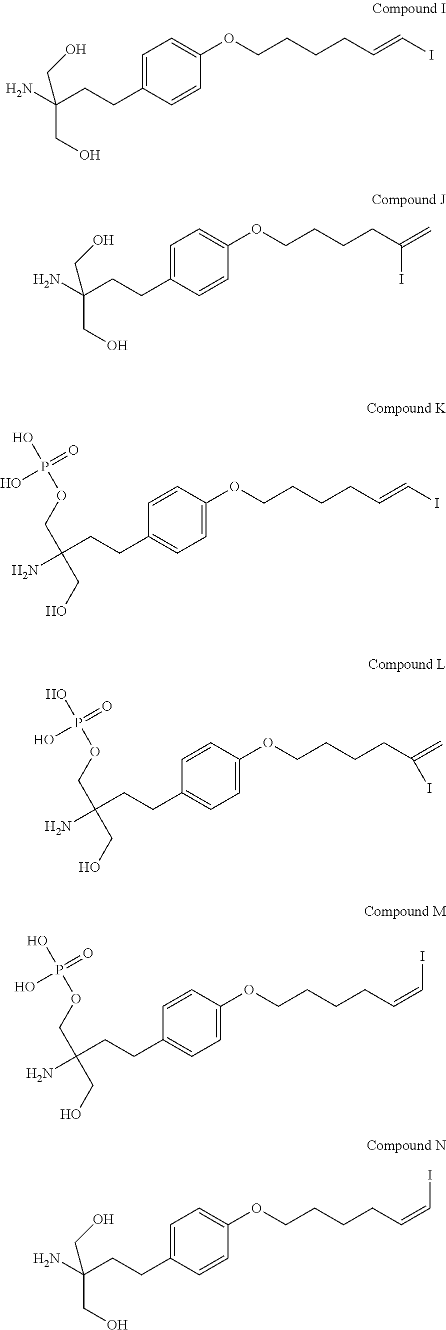

In the above exemplified compounds, the atom I may be substituted with, e.g. replaced by, any one of ¹²³I, ¹²⁵I, ¹²⁴I, ¹³¹I, ⁷⁵Br or ⁷⁶Br, for example by ¹²³I, ¹²⁵I, ¹²⁴ or ¹³¹I, for example by ⁷⁵Br or ⁷⁶Br, or for example by ¹²³I or ¹²⁴I. In which cases the compounds are radiolabelled derivatives of FTY720.

Process

Compounds of formula Ib, e.g. compounds I to N are obtained according to Process 1 which is summarized as follow.

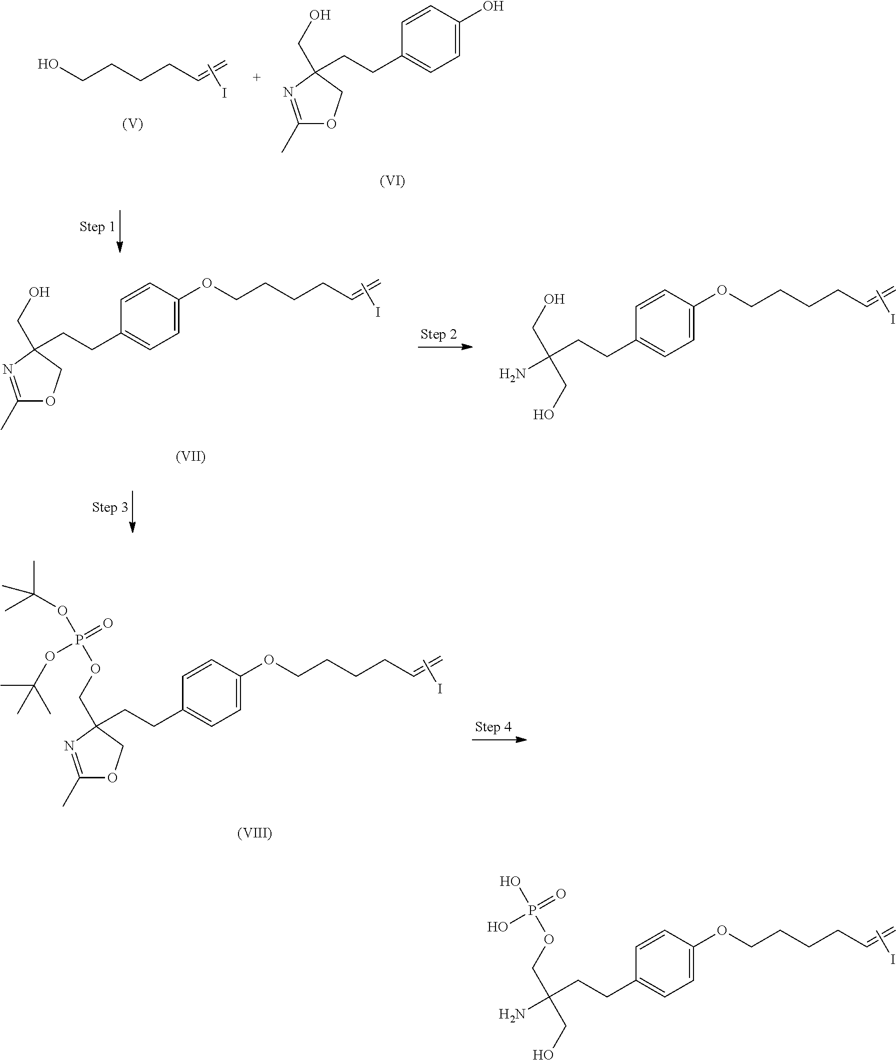

The processes are described in more detail below.

Step 1

A compound of formula (VII) is obtained by reacting a compound of formula (V) with a compound a compound of formula (VI) in the presence of suitable coupling reagents e.g. DIAD or DEAD and PPh₃, in the presence of a solvent or a mixture of solvents, e.g. dioxane, THF.

Step 2

A compound of formula I, J or N is obtained by reacting a compound of formula (VII) in the presence of a suitable acid e.g. concentrated hydrochloric acid, concentrated sulfuric acid or trifluoroacetic acid, in the presence of a solvent, e.g. dioxane, EtOH or MeOH

Step 3

A compound of formula (VIII) is obtained by reacting a compound of formula (VII) with a phosphorylating agent e.g. a phosphorochloridate, diphenylchlorphosphate, cyanoethylphosphate, a phosphoramidite such as di-tertbutyldiethylphosphoramidite, in the presence of a solvent or a mixture of solvents, e.g. DCM, THF or dioxane followed by an oxidative reaction with an oxidizing agent e.g. H₂O₂.

Step 4

A compound of formula K, L or M is obtained by reacting a compound of formula (VIII) in the presence of a suitable acid e.g. concentrated hydrochloric acid, concentrated sulfuric acid or trifluoroacetic acid, in the presence of a solvent or a mixture of solvents, e.g. dioxane, EtOH or MeOH

Compounds of formula Ia, e.g. compounds A, B, D, E, F and H are obtained according to Process 2 which is summarized by the following scheme.

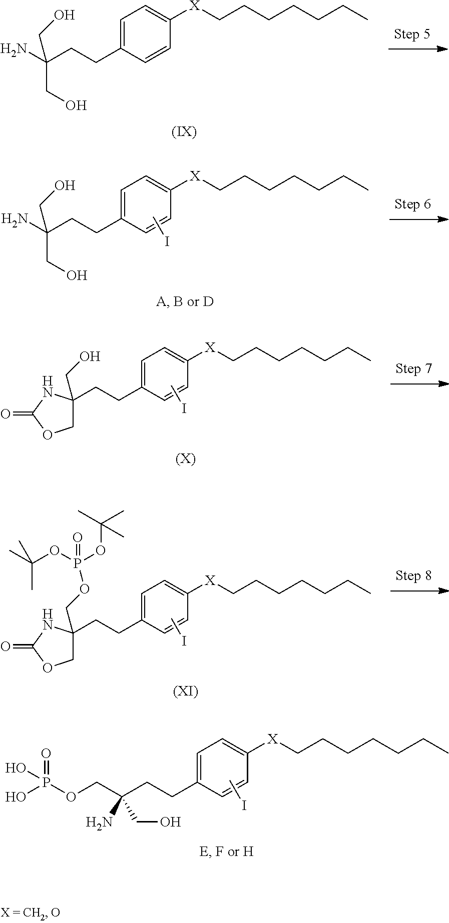

Step 5

A compound of formula A, B or D is obtained by reacting a compound of formula (IX) in the presence of iodine, a suitable acid e.g. concentrated hydrochloric acid, concentrated sulfuric acid or trifluoroacetic acid, in the presence of a solvent or a mixture of solvents, e.g. CH₃CN, dioxane, EtOH or MeOH

Step 6

A compound of formula (X) is obtained by reacting a compound of formula A, B or D in the presence of benzyl chloroformate, a suitable base e.g. sodium hydroxide in the presence of a solvent or a mixture of solvents, e.g. CH₃CN, dioxane, EtOH or MeOH

Step 7

A compound of formula (XI) is obtained by reacting a compound of formula (X) with a phosphorylating agent, e.g. a phosphorochloridate, diphenylchlorphosphate, cyanoethylphosphate, a phosphoramidite such as di-tertbutyldiethylphosphoramidite, in the presence of a solvent or a mixture of solvents, e.g. DCM, THF or dioxane, followed by an oxidative reaction with an oxidizing agent e.g. H₂O₂.

Step 8

A compound of formula E, F or H is obtained by reacting a compound of formula (XI) in the presence of a suitable acid e.g. concentrated hydrochloric acid, concentrated sulfuric acid or trifluoroacetic acid, in the presence of a solvent or a mixture of solvents, e.g. dioxane, EtOH or MeOH.

The present invention also provides a radiolabelled compound of formula (IIIa) or (IVa).

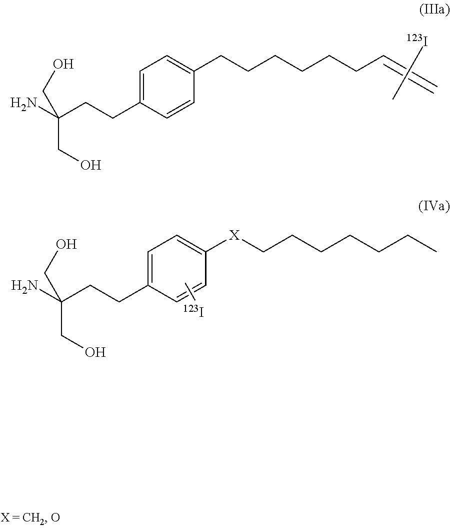

The compounds of formula (IIIa) and (IVa) are obtained by reacting the corresponding stannane, or borane in the presence of a source of radioactive alkali metal halide

The labeling of the compounds of formula (IIIa) and (IVa) can be obtained by several techniques. For example, it can be carried out by reacting a trialkylstannane precursor, a borane precursor or a boronic precursor of the compound of formula (IIIa), (IVa) and an alkali metal halide, such as Na¹²³I, Na¹²⁴I, Na¹²⁵I, Na¹³¹I, Na⁷⁵Br or Na⁷⁶Br in the presence of an oxidizing agent, such as chloramines-T, peracetic acid or aqueous hydrogen peroxide solution, and an acid, such as hydrochloric acid, acetic acid or an acidic buffer, preferably at ambient temperature and in an appropriate solvent. The labeling can also take place by exchange in an acidic medium between the nonradioactive iodinated molecule and a radioactive alkali metal halide.

The inventors approach was based on the use of organoboron compounds (boronic acid, pinacol-boronate, trifluoroboronate and neopentyl boronate) as precursors to radiohalogenated iodo-FTY720 derivatives.

RadiolabelledRadiolabelled compounds of formula Ia, e.g. compounds A, B and D may be obtained according to Process 3 which is summarized by the following scheme.

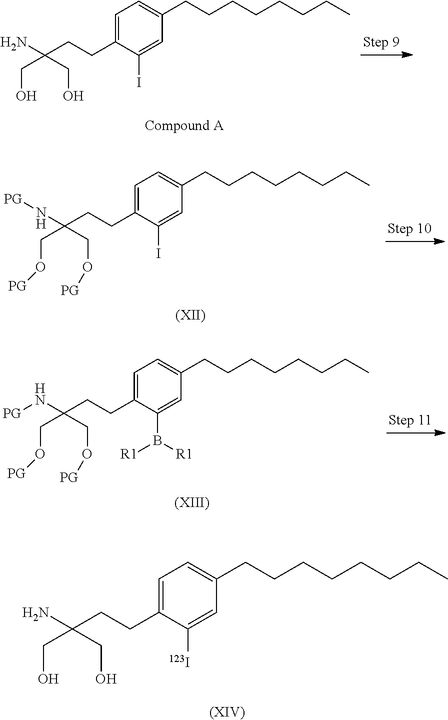

Step 9

A compound of formula (XII) is obtained by reacting compound A in the presence of suitable protecting groups as described by Greene et al (Protective groups in Organic Synthesis, Wiley), e.g. alkyl t-butyl carbonate, acetonide, acetate ester, in the presence of a suitable base e.g. sodium hydroxide, in the presence of a suitable solvent or a mixture of solvents, e.g. DMF, DMSO, dioxane.

Step 10

A compound of formula (XIII) is obtained by reacting a compound of formula (XII) via a cross-coupling reaction of a suitable diboron compound e.g. bis(pinacolato)diboron, bis(neopentyl)diboron in the presence of a suitable palladium catalyst e.g. PdCl₂(PPh₃)₂₋₂PPh₃, PdCl₂(dppf), in the presence of a suitable base e.g. K₂CO₃, KOAc in the presence of a suitable solvent or a mixture of solvents e.g. dioxane, DMSO and subsequently by hydrolysis in the presence of a suitable acid e.g. hydrochloric acid or potassium hydrogen fluoride.

Step 11

A compound of formula (XIV) is obtained by reacting a compound of formula (XIII) in the presence of a source of iodine e.g. NaI, in the presence of a suitable oxydazing agent, e.g. chloramines-T, peracetic acid or aqueous hydrogen peroxide solution, in the presence a suitable base, in the presence of a suitable solvent or a mixture of solvent e.g. H₂O, THF, dioxane and subsequently by removal of the protecting group with a suitable acid e.g. hydrochloric acid, trifluoroacetic acid.

RadiolabelledRadiolabelled compounds of formula Ib, e.g. compounds I, J and N are obtained following an analogous synthetic scheme to Process 3.

Diseases

As hereinabove defined, “diseases or disorders where S1P receptors expression is affected” refers to diseases or disorders resulting in an imbalance or dysfunction of one or more S1P receptors, e.g. of anyone of S1P1, S1P4, S1P5 and S1P3 receptors.

For example, such diseases include inflammatory diseases, autoimmune diseases, demyelinating diseases, neurodegenerative diseases, brain diseases, cardiovascular diseases, atherosclerosis, cancers, or any disease wherein S1P receptor expression is affected.

As herein defined, autoimmune diseases include, but are not limited to, multiple sclerosis, systemic lupus erythematosus (SLE), arthritis, rheumatoid arthritis, diabetes, (e.g. type I diabetes mellitus, type II adult onset diabetes mellitus), uveitis.

As herein defined, cardiovascular diseases include, but are not limited to, hypertension, heart rate dysregulation.

As herein defined, demyelinating disease, include, but are not limited to, multiple sclerosis, and disorders associated therewith, e.g. optic neuritis and Guillain-Barré syndrome.

As herein defined, neurodegenerative diseases include, but are not limited to, progressive dementia, β-amyloid-related inflammatory diseases, Alzheimer disease, amyloidosis, Lewy Body diseases, Multi-Infarct dementia, Pick's disease or cerebral atherosclerosis.

The present invention is particularly suited for patients affected or suffering from a disease selected from inflammatory diseases, autoimmune diseases, demyelinating diseases, neurodegenerative diseases, brain diseases, cardiovascular diseases, atherosclerosis, and cancers, e.g. from a disease selected from inflammatory diseases, autoimmune diseases, demyelinating diseases, neurodegenerative diseases, and brain diseases.

In another embodiment the invention is addressed to patients suspected of suffering from such a disease.

Diagnostic and Imaging Uses

As previously stated, the present invention provides novel FTY720 derivatives as herein above defined, e.g. iodinated or brominated FTY720 derivatives, e.g. compounds of formula I, Ia or Ib, which can be used as myelin sheet or S1P receptors tracers for in vitro and in vivo imaging applications using an appropriate imaging instrument, in particular for brain or spinal cord imaging.

There are several differences between PET/SPECT imaging and established MRI techniques, and both methods can be considered complementary. While MRI can be used for imaging lesions in e.g. multiple sclerosis, it has limitations that PET/SPECT tracers can overcome. For instance, MRI methods are based on tissue water content and do not clearly differentiate T2-weighted MRI hyperintensities resulting from e.g. neurodegeneration following stroke, hemorrhage, or inflammatory processes. In contrast, the compounds of the invention allow specific myelin imaging, either following insertion in the myelin sheet or by binding to S1P receptors expressed in myelin.

In addition, PET/SPECT imaging does not require the use of a Gd-based contrast-enhancing agent, e.g. to differentiate between chronic and acute (or active) lesions. Finally, MRI is contra-indicated in patients with metallic implants, cardiac pacemakers, cochlear implants, older-generation aneurysm clips, implanted stimulators or metallic foreign bodies in the eye.

As herein above defined, “imaging instrument” refers to an instrument that can detect the radiations emitted from radiotracers administered to living subjects and may reconstruct the information obtained to provide planar and tomographic images. Such images may reveal the distribution and/or concentration of the radiotracer as a function of time. Preferably, the “imaging instrument” of the present invention refers, but are not limited to, positron emission tomography (PET) or single photon emission computed tomography (SPECT).

These tracers can be used for imaging S1P receptors in tissue sections in vitro or in vivo, in particular in the brain, for example for analysing the receptor occupancy of compounds having an affinity for the S1P receptors, e.g. for the S1P1, S1P3, S1P4 and/or S1P5 receptors. The compounds of the invention are useful, for instance, for determining the levels of S1P receptors inhibition of a drug acting on such receptors.

They can be used to evaluate the potential therapeutic application of such compounds. They can be useful for monitoring the effectiveness of pharmaco-therapies of such diseases

These tracers can be used for diagnosing appearance or examining a S1P related disease or disorder as herein defined, for example autoimmune or demyelinating diseases, such as multiple sclerosis.

They can be used to evaluate whether a patient is susceptible to be treated with a drug acting through S1P receptors interaction, e.g. to be treated with FTY720.

In a series of embodiments, the present invention provides
1.1 Derivatives of FTY720 as defined hereinabove, e.g. iodo or bromo derivatives of FTY720, e.g. compounds of formula I, Ia or Ib, e.g. compounds of formula Ia and Ib; or corresponding radiolabelledradiolabelled derivatives thereof, e.g. radiolabelledradiolabelled compounds of formula I, Ia or Ib, e.g. compounds of formula Ia or Ib comprising at least one atom selected from ¹²³I, ¹²⁴I, ¹²⁵I, ¹³¹I, ⁷⁵Br and ⁷⁶Br. Preferably, Compounds A to H, e.g. Compounds A, C, E and/or G, or corresponding radiolabelledradiolabelled compounds. Preferably compounds selected from any one of Compound A and Compound E and the corresponding radiolabelledradiolabelled compounds.
1.2 Derivatives of FTY720 as defined hereinabove, e.g. compounds of formula I, Ia or Ib, e.g. compounds of formula Ia or Ib; or the corresponding radiolabelled compounds, e.g. radiolabelled compounds of formula I, Ia and Ib, e.g. radiolabelled compounds of formula Ia or Ib, as markers for labeling S1P receptors, e.g. for labeling at least one of S1P1, S1P3, S1P4 and S1P5 receptors, e.g. for labeling S1P1 and/or S1P5 receptors.
1.3 Derivatives of FTY720 as defined hereinabove, e.g. compounds of formula I, Ia or Ib, e.g. compounds of formula Ia or Ib; and the corresponding radiolabelled compounds, e.g. radiolabelled compounds of formula I, Ia or Ib, e.g. radiolabelled compounds of formula Ia or Ib, as markers for diseases or disorders where S1P receptor expression is altered, for example a disease selected from an autoimmune disease, neurodegenerative disease, brain disease, or demyelinating disease, e.g. multiple sclerosis.
2.1 Use of a derivative of FTY720 as defined hereinabove, e.g. an iodo or bromo derivative of FTY720 as defined hereinabove, e.g. a compound of formula I, Ia or Ib, e.g. a compound of formula Ia or Ib; or the corresponding radiolabelled compounds as hereinabove defined, e.g. a radiolabelled compound of formula Ia or Ib, as radiotracer, e.g. for positron emission tomography (PET) or single photon emission computed tomography (SPECT).
2.2 Use of a derivative of FTY720 as defined hereinabove, e.g. an iodo or bromo derivative of FTY720 as defined hereinabove, e.g. a compound of formula I, Ia or Ib, or the corresponding radiolabelled compounds, e.g. the radiolabelled compound of formula Ia or Ib, for diagnosing diseases or disorders where S1P receptor expression is altered, for example a disease selected from an autoimmune disease, neurodegenerative disease, brain disease or demyelinating disease, for example multiple sclerosis.
3. Use of a derivative of FTY720 as defined hereinabove, e.g. an iodo or bromo derivative of FTY720 as defined hereinabove, e.g. a compounds of formula I, Ia or Ib, e.g. a compound of formula Ia or Ib; or the corresponding radiolabelled compounds, e.g. the radiolabelled compound of formula Ia or Ib, for in vitro or in vivo imaging applications, e.g. brain or spinal cord imaging, e.g. with positron emission tomography (PET) or single photon emission computed tomography (SPECT), for example in a patient suffering from or suspected of having an autoimmune disease, neurodegenerative disease, brain disease or demyelinating disease, for example multiple sclerosis.
4.1 A method to diagnose the appearance of a diseases or disorders where S1P receptor expression is altered, for example an autoimmune disease or demyelinating disease, in a subject, wherein said method comprises using a radiolabelled derivative of FTY720 as defined hereinabove, e.g. a radiolabelled iodo or bromo derivative of FTY720 as defined hereinabove, e.g. a compound of formula Ia or Ib, as hereinabove defined.
4.2 A method to diagnose in a subject a disease or disorder wherein the S1P receptor expression is altered using PET or SPECT, for example to diagnose an inflammatory disease, autoimmune disease, neurodegenerative disease, brain disease or demyelinating disease, wherein said method comprises radio-labeling a derivative of FTY720 as defined hereinabove, e.g. an iodo or bromo derivative of FTY720 as defined hereinabove, e.g. a compound of formula I, Ia or Ib, e.g. a compound of formula Ia or Ib, as hereinabove defined.
4.3 A method to diagnose the appearance of a disease or disorder where S1P receptor expression is altered, for example an inflammatory disease, autoimmune disease, neurodegenerative disease, brain disease or demyelinating disease, in a subject, wherein said method comprises
   a) administering a radiolabelled derivative of FTY720 as defined hereinabove, a radiolabelled iodo or bromo derivative of FTY720 as defined hereinabove, e.g. a radiolabelled compound of formula I, Ia or Ib, e.g. a compound of formula I, Ia or Ib, as hereinabove defined, to the subject, and
   b) detecting or measuring the radiation emitted from the radiolabelled compound with an appropriate imaging instrument, e.g. positron emission tomography (PET) or single photon emission computed tomography (SPECT), and optionally
   c) reconstructing the information obtained in step b) to provide planar and tomographic images which reveal the distribution and/or concentration of the radiolabelled compound as a function of time.
4.4 A method to diagnose the appearance of a disease or disorder where S1P receptor expression is altered, for example an autoimmune disease, neurodegenerative disease, brain disease or demyelinating disease, in a subject, as defined under point 4.3. above, comprising before step a)
   a step a1) of preparing a radiolabelled derivative of FTY720 as defined hereinabove, e.g. introducing an atom selected from the group consisting of ¹²³I, ¹²⁴I, ¹²⁵I, ¹³¹I, ⁷⁵Br and ⁷⁶Br, e.g. ¹²³I or ¹²⁴I, into an FTY720 derivative.
   Step a1) may consist of preparing a compound of formula I, Ia or Ib containing one iodine or bromo atom which is radioactive, e.g. containing one atom selected from ¹²³I, ¹²⁵I, ¹²⁴I, ¹³¹I, ⁷⁵Br and ⁷⁶Br, e.g. selected from ¹²³I and ¹²⁴I.
5.1 A method to predict if a patient suffering from a disease or disorder selected from an inflammatory disease, autoimmune disease, demyelinating disease, and brain disease, will respond to a compound acting as a S1P receptor modulator, e.g. a S1P receptor agonist, e.g. FTY720, comprising:
   a) administering a radiolabelled iodo derivative of FTY720 as defined hereinabove, e.g. a radiolabelled compound of formula Ia and Ib containing at least one radioactive iodine atom, to a patient, and
   b) detecting or measuring the radiation emitted from the radiolabelled compound with an appropriate imaging instrument, e.g. positron emission tomography (PET) or single photon emission computed tomography (SPECT).
5.2 A method to predict which patients will respond to a compounds acting as a SW receptor modulator, e.g. a S1P receptor agonist, e.g. FTY720, as defined under point 5.1. above, comprising before step a)
   a step a1) of preparing a radiolabelled derivative of FTY720 as hereinabove defined, e.g. introducing an atom selected from the group consisting of ¹²³I, ¹²⁴I, ¹²⁵I, ¹³¹I, ⁷⁵Br or ⁷⁶Br, e.g. ¹²³I or ¹²⁴I, into a FTY720 derivative of the invention, e.g. into a compound of formula I, Ia or Ib. Step a1) may consist of preparing a compound of formula I, Ia or Ib, e.g. of formula Ia or Ib, containing one iodine or bromo atom which is radioactive, e.g. containing at least one atom selected from ¹²³I, ¹²⁵I, ¹²⁴I, ¹³¹I, ⁷⁵Br and ⁷⁶Br, e.g. containing at least one atom selected from ¹²³I and ¹²⁴I.
5.3 A method to estimate the distribution of FTY720 in specific patient populations, e.g. the brain distribution, comprising a step of
   a) preparing a radiolabelled derivative of FTY720 as hereinabove defined, e.g. introducing an atom selected from the group consisting of ¹²³I, ¹²⁴I, ¹²⁵I, ¹³¹I, ⁷⁵Br or ⁷⁶Br, e.g. ¹²³I or ¹²⁴I, into a FTY720 derivative of the invention, e.g. into a compound of formula I, Ia or Ib;
   b) administering said radiolabelled compound to a population of patients, e.g. affected of a disease or disorder selected from an inflammatory disease, autoimmune disease, demyelinating disease and brain disease, or at risk of being affected by such a disease, and
   c) visualizing the distribution of FTY720 in the myelin or brain of such patients. The step of preparing a radiolabelled derivative may consist of preparing a compound of formula I, Ia or Ib, e.g. of formula Ia or Ib, containing one iodine or bromo atom which is radioactive, e.g. containing at least one atom selected from ¹²³I, ¹²⁵I, ¹²⁴I, ¹³¹I, ⁷⁵Br and ⁷⁶Br, e.g. containing at least one atom selected from ¹²³I and ¹²⁴I.
6.1 Use of a derivative of FTY720 as defined hereinabove, e.g. an iodo or bromo derivative of FTY720 as defined hereinabove, e.g. a compound of formula I, Ia or Ib, or the corresponding radiolabelled compounds thereof as hereinabove defined, e.g. a compound of formula I, Ia or Ib containing at least one atom selected from ¹²³I, ¹²⁵I, ¹²⁴I, ¹³¹I, ⁷⁵Br and ⁷⁶Br, e.g. containing at least one atom selected from ¹²³I and ¹²⁴I, for monitoring the effectiveness of a pharmacotherapy of a disease or disorder where S1P receptor expression is altered, for example an inflammatory disease, autoimmune disease, demyelinating disease, neurodegenerative disease, or brain disease.
6.2 Use of a derivative of FTY720 as defined hereinabove, e.g. an iodo or bromo derivative of FTY720, e.g. a compound of formula Ia or Ib, or the corresponding radiolabelled compound thereof, as hereinabove defined, e.g. a compound of formula I, Ia or Ib containing at least one atom selected from ¹²³I, ¹²⁵I, ¹²⁴I, ¹³¹I, ⁷⁵Br and ⁷⁶Br, e.g. containing at least one atom selected from ¹²³I and ¹²⁴I, as a tracer for imaging myelin and/or visualizing brain or spinal cord imaging in a subject, wherein said method comprises
   a) administering a radiolabelled derivative of FTY720 as defined hereinabove, a radiolabelled iodo or bromo derivative of FTY720, e.g. a radiolabelled compound of formula I, Ia or Ib, as hereinabove defined, to the subject, and
   b) detecting or measuring the radiation emitted from the radiolabelled compound with an appropriate imaging instrument, e.g. positron emission tomography (PET) or single photon emission computed tomography (SPECT), and optionally
   c) reconstructing the information obtained in step b) to provide planar and tomographic images which reveal the distribution and/or concentration of the radiolabelled compound as a function of time.
6.3 Use as defined under point 6.2. above, comprising before step a) a step of preparing a radiolabel derivative of FTY720, e.g. introducing an atom selected from the group consisting of ¹²³I, ¹²⁴I, ¹²⁵I, ¹³¹I, ⁷⁵Br and ⁷⁶Br, e.g. ¹²³I or ¹²⁴I, into a FTY720 derivative of the invention.
6.4 Use as defined under point 6.2. above, comprising before step a) a step of preparing a compound of formula I, Ia or Ib, e.g. of formula Ia or Ib, containing at least one atom selected from ¹²³I, ¹²⁵I, ¹²⁴I, ¹³¹I, ⁷⁵Br and ⁷⁶Br, e.g. containing at least one atom selected from ¹²³I and ¹²⁴I.
6.5 Use as defined under point 6.2. or 6.4 above using a positron emission tomography (PET) or single photon emission computed tomography (SPECT).
6.6 Use as defined under points 6.2. to 6.5 above in a patient suffering from, suspected of having, or at risk of suffering from an autoimmune disease, neurodegenerative disease, brain disease or demyelinating disease, for example multiple sclerosis.
6.7 Use of a derivative of FTY720 as hereinabove defined, e.g. an iodo or bromo derivative of FTY720 as hereinabove defined, e.g. a compound of formula I, Ia or Ib, or the corresponding radiolabelled compounds thereof as hereinabove defined, to diagnose the appearance of a disease or disorder where S1P receptor expression is altered, for example an autoimmune disease or demyelinating disease, e.g. multiple sclerosis.
6.8 Use of a derivative of FTY720 as hereinabove defined, e.g. an iodo or bromo derivative of FTY720 as hereinabove defined, e.g. a compound of formula I, Ia or Ib, or the corresponding radiolabelled compounds thereof as hereinabove defined, to diagnose the appearance of a diseases or disorders as according to a method defined above under point 4.3 or 4.4.
6.9 Use of a derivative of FTY720 as hereinabove defined, e.g. an iodo or bromo derivative of FTY720 as hereinabove defined, e.g. a compound of formula I, Ia or Ib, or the corresponding radiolabelled compounds thereof, to predict which patients will respond to a compounds acting as a S1P receptor modulator, e.g. to FTY720.
6.10 Use of a derivative of FTY720 as hereinabove defined, e.g. an iodo or bromo derivative of FTY720 as hereinabove defined, e.g. a compound of formula Ia or Ib, or the corresponding radiolabelled compounds thereof, to estimate the distribution of FTY720 in specific patient populations, e.g. the brain distribution.
7. A method of brain imaging or myelin imaging, comprising administering to a subject an effective amount of a radiolabelled iodo or bromo derivative of FTY720 as defined hereinabove, e.g. a radiolabelled compound of formula I, Ia or Ib, e.g. a radiolabelled compound of formula Ia or Ib.
8. A composition which comprises a compound of the invention, e.g. a derivative of FTY720 as hereinabove defined, e.g. an iodo or bromo derivative of FTY720 as hereinabove defined, e.g. a compound of formula I, Ia or Ib, or the corresponding radiolabelled compound thereof, and a pharmaceutically acceptable carrier or excipient.
9. Use of a radiolabelled compound of the invention, e.g. a radiolabelled compound of formula I, Ia or Ib, e.g. a radiolabelled compound of formula Ia or Ib; or a composition as defined under 8; for labeling histopathological structures containing S1P receptors, e.g. at least one of S1P1, S1P3, S1P4 and S1P5 receptors, in vitro or in vivo.

For example, there is provided the use of a radiolabelled compound of formula I, Ia or Ib, e.g. a compound selected from a radiolabelled Compound A to M, e.g. a radiolabelled Compound A, C, E or G, for performing an in vitro autoradiography, and determining the distribution of the S1P receptors on tissue section. Autoradiography may be done by Quantitative whole Body Autoradiography (QWBA).

As used herein, “pharmaceutically acceptable carrier” includes any and all solvents, dispersion media, coatings, antibacterial and antifungal agents, isotonic agents, absorption delaying agents and the like. The use of such media and agents for pharmaceutically active substance is well known in the art. The compound of the invention may be administered to a patient in an appropriate diluent or adjuvant, or in an appropriate carrier such as human serum albumin or liposomes. Pharmaceutically acceptable diluent include saline and aqueous buffer solutions. Adjuvants may include resocinols, non-ionic surfactants such as polyoxyethylene oleyl ether and hexadecyl polyethylene ether.

In one embodiment of the invention, the compound of the invention, its enantiomer, stereoisomer, racemate or pharmaceutically acceptable salt, e.g. the radiolabelled compound of the invention, is administered parentally as injections (intravenous, intramuscular or subcutaneous). The compound, its enantiomer, stereoisomer, racemate or pharmaceutically acceptable salt, may be formulated as a sterile, pyrogen-free, parenterally acceptable aqueous solution. The preparation of such parenterally acceptable solutions, having due regard to pH, isotonicity, stability, and the like, is known to the person skilled in the art. Certain pharmaceutical compositions suitable for parenteral administration include a radiolabelled compound of the invention in combination with one or more pharmaceutically acceptable sterile powders which may be reconstituted into sterile injectable solutions or dispersions just prior to use. The pharmaceutical compositions may also contain antioxidants, buffers, bacteriostats, solutes which render the formulation isotonic with the blood of the intended recipient, or suspending or thickening agents. A formulation for injection may contain, in addition to the radiolabelled compound of the invention, an isotonic vehicle such as sodium chloride solution, Ringer's solution, dextrose solution, dextrose and sodium chloride solution, lactated Ringer's solution, dextran solution, sorbitol solution, a solution containing polyvinyl alcohol, or an osmotically balanced solution including a surfactant and a viscosity-enhancing agent, or other vehicle as known in the art. The formulations may also contain stabilizers, preservatives, buffers, antioxidants, or other additives known to those skilled in the art.

An effective amount of the compound d of the invention may be combined with a pharmaceutically acceptable carrier for use in imaging studies.

As herein defined “an effective amount” refers to an amount sufficient to yield an acceptable image using adequate method and available equipment, e.g. PET or SPECT.

The effective amount may be administered in more than one administration.

The effective amount may vary according to factors such as the nature and severity of the condition being treated, the disease to be diagnosed or the state of the disease to be diagnosed, on the nature of therapeutic treatments which the patient has undergone, the degree of susceptibility of the patient, his age, sex, weight, and idiosyncratic responses of the patient as well as dosimetry.

The effective amount may vary depending on the used instrument and film-related factors. Choice of the effective amount and optimization of such factors are well known to the person skilled in the art. Ultimately, the attending physician will decide the amount of compound to administer to each individual patient and the duration of the imaging study.

For example, less than 1 microgram of the radiolabelled compound of the invention, e.g. radiolabelled compound of formula I, Ia or Ib, e.g. a compound selected from radiolabelled Compounds A to M, e.g. radiolabelled Compound A, C, E or G, is administered, e.g. for diagnostic or therapeutic purposes. Examples of effective amount of the radiolabelled compound of the invention to be administered in a method of the invention as herein defined, include about 100 picograms to about 10 micrograms, e.g. about 80 picograms to about 15 micrograms, e.g. about 50 picograms to about 20 micrograms, e.g. about 30 picograms to about 30 micrograms, e.g. about 20 picograms to about 35 micrograms, e.g. about 10 picograms to about 40 micrograms.

For example, the effective amount of the radiolabelled compound of the invention, e.g. radiolabelled compound of formula I, Ia or Ib, may be about 100 picograms, about 50 picograms, about 30 picograms, about 20 picograms, about 10 picograms, about 1 picogram, about 100 micrograms, about 80 micrograms, about 50 micrograms, about 40 micrograms, about 30 micrograms, about 20 micrograms, about 10 micrograms, about 5 micrograms, about 1 microgram.

In another embodiment, the radiolabelled compound of the invention, e.g. radiolabelled compound of formula I, Ia or Ib, may be administered as from about 0.1 to about 10 mCi, about 0.5 to about 80 mCi, about 1 to about 50 mCi, about 1 to about 100 mCi.

Such ranges and amounts are particularly suitable when administering the radiolabelled compounds of the invention, e.g. radiolabelled compound of formula I, Ia or Ib, e.g. a compound selected from radiolabelled Compounds A to M, e.g. radiolabelled Compound A, C, E or G, as markers, e.g. imaging or diagnostic agents according to the method described hereinabove, or in a kit as described below.

In another aspect, a kit is provided which includes a radiolabelled compound of the invention, its enantiomer, stereoisomer, racemate or pharmaceutically acceptable salt, as described above, in combination with a pharmaceutically acceptable solution containing a carrier such as human serum albumin or an auxiliary molecule such as mannitol or glaciate. Human serum albumin for use in the kit may be made in any way, for example, through purification of the protein from human serum or through recombinant expression of a vector containing a gene encoding human serum albumin. Other substances may also be used as carriers, for example, detergents, dilute alcohols, carbohydrates, and the like.

In one embodiment, a kit may contain from 1 to about 50 mCi of a radiolabelled compound of the invention, its enantiomer, stereoisomer, racemate or pharmaceutically acceptable salt.

In a specific embodiment of the invention, a kit may contain the unlabeled fatty acid stereoisomer which has been covalently or non-covalently combined with a chelating agent, and an auxiliary molecule such as mannitol, gluconate, and the like. The unlabeled fatty acid stereoisomer/chelating agent may be provided in solution or in lyophilized form. The kit may also include other components which facilitate practice of the described methods. For example, buffers, syringes, film, instructions, and the like may optionally be included as components of the kits of the disclosure.

Therapeutic Use

The compounds of the invention may be used as therapeutic agent for treating or preventing a disease or disorder where S1P receptor expression is altered as hereinabove defined, for example an autoimmune disease or demyelinating disease, for example multiple sclerosis.

The terms “treatment” or “therapy” (especially of a disease or disorder where S1P receptor expression is altered) refer to the prophylactic or preferably therapeutic (including but not limited to palliative, curing, symptom-alleviating, symptom-reducing) treatment of said diseases, especially of the diseases mentioned above.

Furthermore there is provided:
10. A method for treating or preventing a diseases or disorders where S1P receptor expression is altered, e.g. an inflammatory disease, autoimmune disease, neurodegenerative disease, brain disease, or demyelinating disease in a patient in need thereof, such a method comprising administering an iodo or bromo derivative of FTY720 as hereinabove defined, e.g. a compound of formula I, Ia or Ib, or the corresponding radiolabelled compound thereof, as hereinabove defined, or a pharmaceutically acceptable salt thereof.

The following non-limiting Examples illustrate the invention.

A list of Abbreviations used is given below.

Boc tert-butyloxycarbonyl

CH₃CN acetonitrile

DCC Dicyclohexylcarbodiimide

DCE dichloroethane

DCM dichloromethane

DMF N,N′-dimethylformamide

EtOAc ethylacetate

EtOH ethanol

Et₂O diethyl ether

h hours

HPLC high pressure liquid chromatography

K₂CO₃ potassium carbonate

LC liquid chromatography

MeOH methanol

min minutes

mL milliliter

mmol millimole

MS mass spectroscopy

NaHCO₃ sodium bicarbonate

NaOH sodium hydroxide

NH₄OH ammonium hydroxide

PG protecting group

Rt retention time (LC/MS)

RT room temperature

TFA Trifluoroacetic acid

THF tetrahydrofuran

LCMS/HPLC Conditions (%=Percent by Volume)

Method A (Rt_{A}=retention time A)

Gilson 331 pumps coupled to a Gilson UV/VIS 152 detector and a Finnigan AQA spectrometer (ESI), a 50 μL loop injection valve and a Waters XTerra MS C18 3.5 μm 4.6×50 mm column running a gradient Water+0.05% TFA/Acetonitrile+0.05% TFA from 95/5 to 10/90 over 8 min with a flux of 1.5 mL/min

Method B (Rt_{B}=retention time B)

Agilent 1100 series; column Waters XBridge C18 2.5 μm; 3×30 mm; gradient: A water+5% acetonitrile+0.5-1.0% HCO₂H/B acetonitrile+0.5-1.0% HCO₂H; 0 min 10B; 1.70 min 95B; 2.40 min: 95B; 2.45 min: 10B; flow 1.2 ml/min; column temperature 50° C.

Method C (Rt_{C}=retention time C)

Thar SFC 200, Chiralpak IC; 30×250 mm; isocratic: CO₂/2-propanol/2-propylamine 75:25:0.25; flow 90 g/min; BPR: 150 bar

Method D (Rt_{C}=retention time D)

HPLC-ZQ2000, column Acquity HSS-T3 1.8 μm; 2.1×50 mm; gradient: A water+5% acetonitrile+0.5-1.0% HCO₂H/B acetonitrile+0.5-1.0% HCO₂H; 0 min 2B; 4.3 min 98B; 5.0 min: 98B; 5.10 min: 2B; 6.0 min: 2B; flow 1.0 ml/min

Preparative HPLC

Gilson Trilution LC

Column: SunFire C18, 30×100 mm, 5 um

Eluent: Water (+0.1% TFA): acetonitrile (+0.1% TFA) from 85/15 to 65/35 in 16 min; flow 50 mL/min.

¹H-NMR Instruments:

Bruker (360 MHz), Varian Mercury (400 MHz); Bruker Advance (600 MHz).

### Example A

### 2-Amino-2-{2-[4-((E)-6-iodo-hex-5-enyloxy)-phenyl]-ethyl}-propane-1,3-diol HCl salt

### Step 1: (4-{2-[4-((E)-6-Iodo-hex-5-enyloxy)-phenyl]-ethyl}-2-methyl-4,5-dihydro-oxazol-4-yl)-methanol

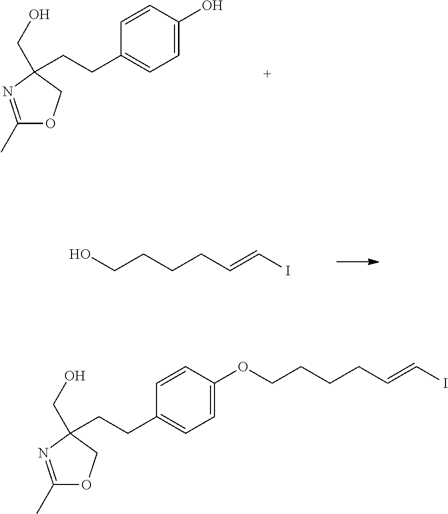

At 0° C., to a mixture of 4-[2-(4-hydroxymethyl-2-methyl-4,5-dihydro-oxazol-4-yl)-ethyl]-phenol (260 mg. 1.1 mmol), (E)-6-iodo-hex-5-en-1-ol (250 mg, 1.0 eq) and triphenylphosphine (290 mg, 1.0 eq) in THF (10 mL) is added DIAD (0.215 mL, 1.0 eq). The resulting mixture is stirred at RT for 18 hours and 24 hours at 50° C. 0.3 equivalent of DIAD and PPh₃ are added and the mixture is stirred for 72 additional hours at 50° C. 0.5 equivalent of DIAD and PPh₃ are added and the mixture is stirred for an additional hour at 50° C. The mixture is partitioned between AcOEt and saturated NH₄Cl. The organic phase is separated, dried over sodium sulfate and concentrated in vacuo to afford a crude beige oil (1.54 g). The crude product is purified by flash chromatography on silica gel using DCM/MeOH (100/0 to 90/10) as solvent system. From the purification, (4-{2-[4-((E)-6-iodo-hex-5-enyloxy)-phenyl]-ethyl}-2-methyl-4,5-dihydro-oxazol-4-yl)-methanol is isolated as colorless oil.

LC/MS: Rt_{A} 4.84 min, m/z: 444.0 [M+H]

¹H NMR (400 MHz, CDCl₃) δ ppm 7.08 (d, 2H); 6.79 (d, 2H); 6.52 (m, 1H); 6.01 (d, 2H); 5.30 (s, 1H); 4.27 (m, 1H); 4.09 (m, 1H); 3.92 (t, 2H); 3.72 (m, 1H); 3.45 (m, 1H); 2.54 (m, 2H); 2.12 (m, 2H); 2.06 (s, 3H); 1.88 (m, 1H); 1.75 (m, 3H); 1.57 (m, 2H).

### Step 2: 2-Amino-2-{2-[4-((E)-6-iodo-hex-5-enyloxy)-phenyl]ethyl}-propane-1,3-diol HCl salt

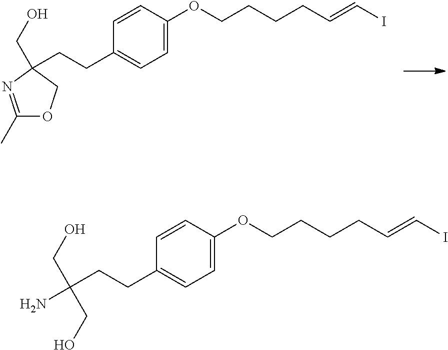

To a solution of (4-{2-[4-((E)-6-iodo-hex-5-enyloxy)-phenyl]-ethyl}-2-methyl-4,5-dihydro-oxazol-4-yl)-methanol (60 mg, 0.135 mmol) in EtOH (2 mL) is added concentrated hydrochloric acid (2.05 mL). The resulting mixture is stirred at 85° C. for 2.5 hours. The solvents are removed in vacuo to afford a beige paste. After precipitation in Et₂O, 2-amino-2-{2-[4-((E)-6-iodo-hex-5-enyloxy)-phenyl]-ethyl}-propane-1,3-diol HCl salt is isolated as a beige powder.

LC/MS: Rt_{A} 4.62 min, m/z: 419.9 [M+H]

¹H NMR (400 MHz, DMSO-d₆) δ ppm 7.80 (bs, 3H); 7.09 (d, 2H); 6.83 (d, 2H); 6.52 (m, 1H); 6.22 (d, 2H); 5.37 (t, 1H); 3.90 (t, 2H); 3.50 (m, 4H); 2.08 (m, 2H); 1.75 (m, 2H); 1.65 (m, 2H); 1.50 (m, 2H).

### Example B

### (R/S)-phosphoric acid mono-{2-amino-2-hydroxymethyl-4-[4-((E)-6-iodo-hex-5-enyloxy)-phenyl]-butyl}ester

### Step 3: (R/S)-phosphoric acid di-tert-butyl ester 4-{2-[4-((E)-6-iodo-hex-5-enyloxy)-phenyl]-ethyl}-2-methyl-4,5-dihydro-oxazol-4-ylmethyl ester

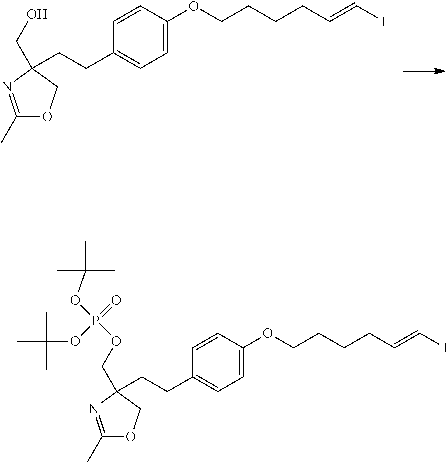

At 0° C., to a solution of (4-{2-[4-((E)-6-iodo-hex-5-enyloxy)-phenyl]-ethyl}-2-methyl-4,5-dihydro-oxazol-4-yl)-methanol (230 mg, 0.52 mmol) in DCM/THF (2 mL/2 mL) is added 1H-tetrazole (182 mg, 5.0 eq) and di-tert-butyldiethylphosphoramidite (0.433 mL, 3.0 eq). The resulting mixture is stirred at RT for 6 hours. Then, H₂O₂ 30% wt. % solution in water (0.159 mL, 10 eq) is added and the mixture is stirred for 1.5 hours at RT. The reaction mixture is quenched by careful addition of a solution of 1N sodium thiosulfate (10 mL). The aqueous phase is extracted with DCM. The organic phases are combined, washed with brine, dried over sodium sulfate and concentrated in vacuo to afford a crude oil (500 mg). The crude oil is purified by flash chromatography on silica gel using DCM/MeOH (100/0 to 90/10) as solvent system. From the purification, (R/S)-phosphoric acid di-tert-butyl ester 4-{2-[4-((E)-6-iodo-hex-5-enyloxy)-phenyl]-ethyl}-2-methyl-4,5-dihydro-oxazol-4-ylmethyl ester (107 mg) is isolated as a clear oil with a purity of around 50% and is used as such for the next step.

LC/MS: Rt_{A} 5.53 min, m/z: 636.1 [M+H]

### Step 4: (R/S)-phosphoric acid mono-{2-amino-2-hydroxymethyl-4-[4-((E)-6-iodo-hex-5-enyloxy)-phenyl]-butyl}ester

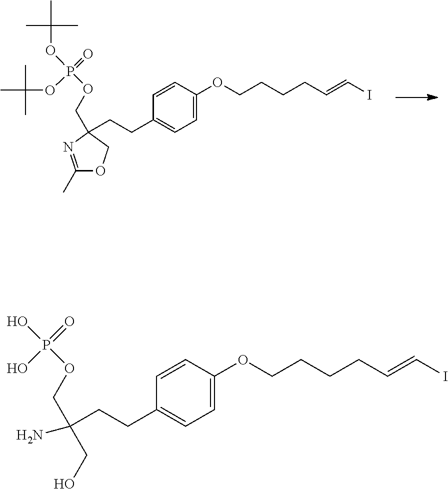

To a solution of (R/S)-phosphoric acid di-tert-butyl ester 4-{2-[4-((E)-6-iodo-hex-5-enyloxy)-phenyl]-ethyl}-2-methyl-4,5-dihydro-oxazol-4-ylmethyl ester (107 mg, 0.168 mmol) in EtOH (2.5 mL) is added concentrated hydrochloric acid (2.56 mL). The resulting mixture is stirred at 85° C. for 2.5 hours. The solvents are removed in vacuo to afford a beige paste. After precipitation in Et₂O, (R/S)-phosphoric acid mono-{2-amino-2-hydroxymethyl-4-[4-((E)-6-iodo-hex-5-enyloxy)-phenyl]-butyl}ester is isolated as a beige powder.

LC/MS: Rt_{A} 4.53 min, m/z: 500.0 [M+H]

¹H NMR (400 MHz, DMSO-d₆) δ ppm 7.09 (d, 2H); 6.82 (d, 2H); 6.52 (m, 1H); 6.22 (d, 1H); 3.89 (m, 4H); 3.53 (m, 2H); 2.08 (m, 2H); 1.78 (m, 2H); 1.65 (m, 2H); 1.49 (m, 2H)

### Example C

### 2-Amino-2-{2-[4-((Z)-6-iodo-hex-5-enyloxy)-phenyl]-ethyl}-propane-1,3-diol TFA salt

### Step 1: (4-{2-[4-((Z)-6-Iodo-hex-5-enyloxy)-phenyl]-ethyl}-2-methyl-4,5-dihydro-oxazol-4-yl)-methanol

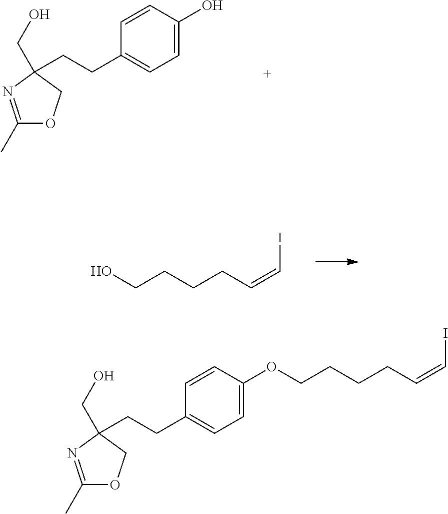

Synthesis analogous to Example A step 1 starting with 4-[2-(4-hydroxymethyl-2-methyl-4,5-dihydro-oxazol-4-yl)-ethyl]-phenol (400 mg. 1.7 mmol), (Z)-6-iodo-hex-5-en-1-ol (250 mg, 1.0 eq). (4-{2-[4-((Z)-6-iodo-hex-5-enyloxy)-phenyl]-ethyl}-2-methyl-4,5-dihydro-oxazol-4-yl)-methanol (403 mg) is isolated as a clear oil.

LC/MS: Rt_{A} 4.76 min, m/z: 443.9 [M+H]

### Step 2: 2-Amino-2-{2-[4-((Z)-6-iodo-hex-5-enyloxy)-phenyl]-ethyl}-propane-1,3-diol TFA salt

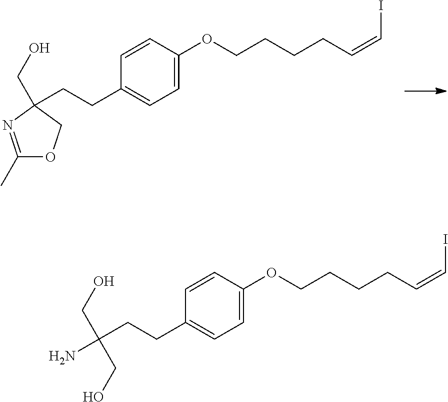

Synthesis analogous to Example A step 2 starting with (4-{2-[4-((Z)-6-iodo-hex-5-enyloxy)-phenyl]-ethyl}-2-methyl-4,5-dihydro-oxazol-4-yl)-methanol. After purification by reverse preparative HPLC, 2-amino-2-{2-[4-((Z)-6-iodo-hex-5-enyloxy)-phenyl]-ethyl}-propane-1,3-diol TFA salt is obtained as a white powder.

LC/MS: Rt_{A} 4.42 min, m/z: 420.0 [M+H]

¹H NMR (400 MHz, DMSO-d₆) δ ppm 7.77 (bs, 3H); 7.08 (d, 2H); 6.84 (d, 2H); 6.40 (m, 1H); 6.29 (m, 1H); 5.40 (t, 1H); 3.92 (t, 2H); 3.50 (m, 4H); 2.13 (m, 2H); 1.72 (m, 4H); 1.53 (m, 2H).

### Example D

### (R/S)-phosphoric acid mono-{2-amino-2-hydroxymethyl-4-[4-((z)-6-iodo-hex-5-enyloxy)-phenyl]-butyl}ester

### Step 3: (R/S)-phosphoric acid di-tert-butyl ester 4-{2-[4-((Z)-6-iodo-hex-5-enyloxy)-phenyl]-ethyl}-2-methyl-4,5-dihydro-oxazol-4-ylmethyl ester

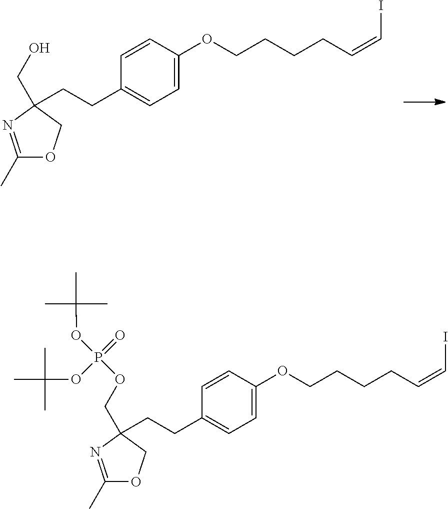

Synthesis analogous to Example B step 3 starting with (4-{2-[4-((Z)-6-iodo-hex-5-enyloxy)-phenyl]-ethyl}-2-methyl-4,5-dihydro-oxazol-4-yl)-methanol. After reaction work-up, (R/S)-phosphoric acid di-tert-butyl ester 4-{2-[4-((Z)-6-iodo-hex-5-enyloxy)-phenyl]-ethyl}-2-methyl-4,5-dihydro-oxazol-4-ylmethyl ester is used as such for the next step.

### Step 4: (R/S)-phosphoric acid mono-{2-amino-2-hydroxymethyl-4-[4-((Z)-6-iodo-hex-5-enyloxy)-phenyl]-butyl}ester

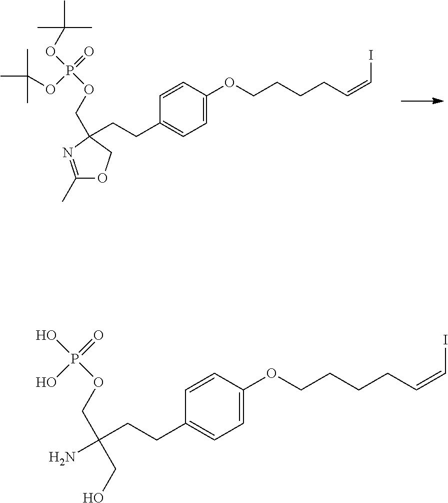

Synthesis analogous to Example B step 4 starting with (R/S)-phosphoric acid di-tert-butyl ester 4-{2-[4-((Z)-6-iodo-hex-5-enyloxy)-phenyl]-ethyl}-2-methyl-4,5-dihydro-oxazol-4-ylmethyl ester. (R/S)-phosphoric acid mono-{2-amino-2-hydroxymethyl-4-[4-((Z)-6-iodo-hex-5-enyloxy)-phenyl]-butyl}ester is isolated as a white powder.

LC/MS: Rt_{A} 4.44 min, m/z: 500.1 [M+H]

¹H NMR (400 MHz, DMSO-d₆) δ ppm 7.09 (d, 2H); 6.82 (d, 2H); 6.40 (m, 1H); 6.30 (m, 1H); 3.91 (m, 3H); 3.80 (m, 2H); 3.52 (m, 2H); 3.33 (bs, 2H); 2.52 (m, 2H); 2.12 (m, 2H); 1.72 (m, 4H); 1.54 (m, 2H)

### Example E

### 2-Amino-2-{2-[4-(5-iodo-hex-5-enyloxy)-phenyl]-ethyl}-propane-1,3-diol HCl salt

### Step 1: (4-{2-[4-(5-Iodo-hex-5-enyloxy)-phenyl]-ethyl}-2-methyl-4,5-dihydro-oxazol-4-yl)-methanol

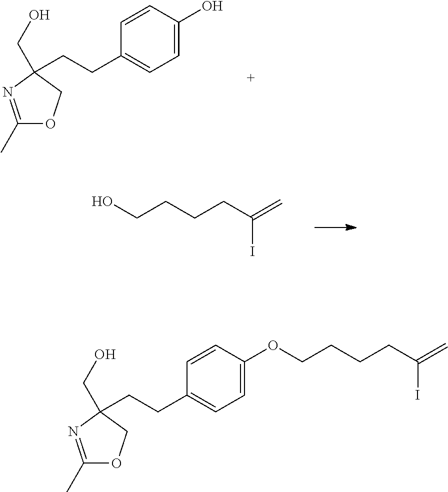

Synthesis analogous to Example A step 1 starting with 4-[2-(4-hydroxymethyl-2-methyl-4,5-dihydro-oxazol-4-yl)-ethyl]-phenol (505 mg. 2.15 mmol), 5-iodo-hex-5-en-1-ol (728 mg, 1.5 eq). (4-{2-[4-(5-iodo-hex-5-enyloxy)-phenyl]-ethyl}-2-methyl-4,5-dihydro-oxazol-4-yl)-methanol is isolated as a clear oil.

LC/MS: Rt_{A} 4.76 min, m/z: 444.0 [M+H]

### Step 2: 2-Amino-2-{2-[4-(5-iodo-hex-5-enyloxy)-phenyl]-ethyl}-propane-1,3-diol HCl salt

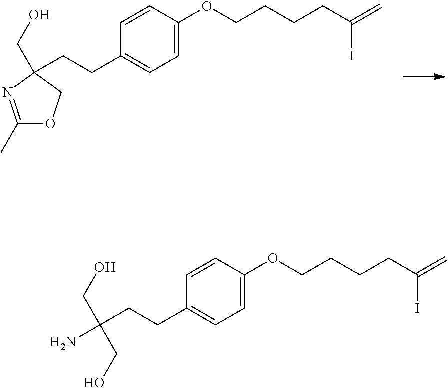

Synthesis analogous to Example A step 2 starting with (4-{2-[4-(5-iodo-hex-5-enyloxy)-phenyl]-ethyl}-2-methyl-4,5-dihydro-oxazol-4-yl)-methanol (63 mg, 0.142 mmol). Reaction is performed in dioxane at 50° C. for 20 hours and then 70° C. for 4 hours. 2-Amino-2-{2-[4-(5-iodo-hex-5-enyloxy)-phenyl]-ethyl}-propane-1,3-diol HCl salt is obtained as a beige powder.

LC/MS: Rt_{A} 4.64 min, m/z: 420.0 [M+H]

¹H NMR (400 MHz, DMSO-d₆) δ ppm 7.77 (bs, 3H); 7.09 (d, 2H); 6.84 (d, 2H); 6.18 (s, 1H); 5.60 (s, 1H); 5.36 (t, 2H); 3.93 (t, 2H); 3.50 (m, 4H); 2.43 (m, 2H); 1.75-1.50 (m, 6H).

### Example F

### (R/S)-Phosphoric acid mono-{2-amino-2-hydroxymethyl-4-[4-(5-iodo-hex-5-enyloxy)-phenyl]-butyl}ester

### Step 3: (R/S)-Phosphoric acid di-tert-butyl ester 4-{2-[4-(5-iodo-hex-5-enyloxy)-phenyl]-ethyl}-2-methyl-4,5-dihydro-oxazol-4-ylmethyl ester

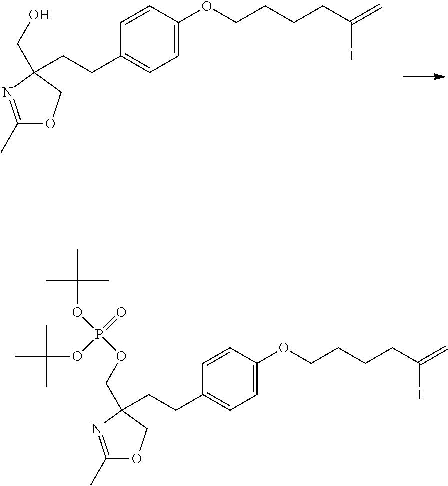

Synthesis analogous to Example B step 3 starting with (4-{2-[4-(5-iodo-hex-5-enyloxy)-phenyl]-ethyl}-2-methyl-4,5-dihydro-oxazol-4-yl)-methanol. After purification by flash chromatography, (R/S)-phosphoric acid di-tert-butyl ester 4-{2-[4-(5-iodo-hex-5-enyloxy)-phenyl]-ethyl}-2-methyl-4,5-dihydro-oxazol-4-ylmethyl ester is used as such for the next step.

### Step 4: (R/S)-Phosphoric acid mono-{2-amino-2-hydroxymethyl-4-[4-(5-iodo-hex-5-enyloxy)-phenyl]-butyl}ester

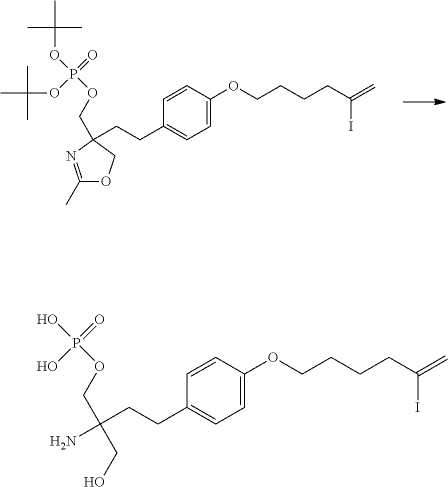

Synthesis analogous to Example 2 step b starting with 4-{2-[4-(5-iodo-hex-5-enyloxy)-phenyl]-ethyl}-2-methyl-4,5-dihydro-oxazol-4-ylmethyl ester. Reaction is performed in dioxane at 50° C. for 4 hours. After purification by reverse preparative HPLC, (R/S)-phosphoric acid mono-{2-amino-2-hydroxymethyl-4-[4-(5-iodo-hex-5-enyloxy)-phenyl]-butyl}ester is isolated as a beige powder.

LC/MS: Rt_{A} 4.45 min, m/z: 499.9 [M+H]

¹H NMR (400 MHz, DMSO-d₆) δ ppm 7.09 (d, 2H); 6.84 (d, 2H); 6.19 (s, 1H); 5.71 (s, 1H); 3.95-3.3.82 (m, 4H); 3.55-3.20 (m, 6H); 2.43 (m, 2H); 1.8-1.5 (m, 6H).

### Examples G and H

### 2-Amino-2-[2-(3-iodo-4-octylphenyl)ethyl]-1,3-propandiol and 2-Amino-2-[2-(2-iodo-4-octylphenyl)ethyl]-1,3-propandiol

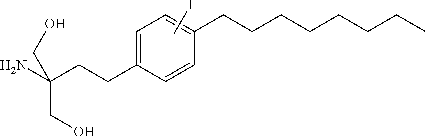

To a solution of FTY720 (3.2 g, 10.4 mmol) in 100 mL wet methylene chloride, silver sulphate (3.25 g, 10.4 mmol) and iodine (2.64 g, 10.41 mmol) are added. Silver trifluoromethanesulfonate (0.13 g, 0.52 mmol) is added at room temperature and the resulting mixture stirred for 18 hours at room temperature. The yellow solid silver iodide is filtered off. The organic phase is washed with 15% aqueous NaHCO₃, dried over sodium sulphate, filtered and evaporated to dryness.

The residue is purified on a silica gel column to yield after drying a mixture of iodo compounds 7 and 8. Purification by chromatography (column: Chiralpak IC, 30×250 mm, mobile phase: CO₂/2-propanol/2-propylamine 75:25:0.25 (isocratic)) afforded the title compounds as white solid.

### Example G

LCMS Rt_{B}=1.28 min, [M]⁺=433.9

SFC Rt_{C}=4.82 min

¹H-NMR (500 MHz, DMSO-d₆) δ ppm 7.62 (d, 1H); 7.01-7.20 (m, 2H); 4.43 (t, 2H); 3.11-3.27 (m, 4H); 2.55-2.63 (m, 2H); 2.45-2.52 (m, 2H); 1.38-1.57 (m, 4H); 1.16-1.35 (m, 12H); 0.76-0.93 (m, 3H)

### Example H

LCMS Rt_{B}=1.29 min, [M]⁺=433.9

SFC Rt_{C}=5.58 min

¹H-NMR (500 MHz, DMSO-d₆) δ ppm 7.56 (d, 1H); 7.06-7.21 (m, 2H); 4.40 (t, 2H); 3.17-3.27 (m, 4H); 2.58-2.64 (m, 2H); 2.41-2.47 (m, 2H); 1.39-1.42 (m, 2H); 1.20-1.31 (m, 12H); 0.77-0.89 (m, 3H)

### Example I

### 2-amino-2-[2-[3-iodo-4-(heptyloxy)phenyl]ethyl]-1,3-propanediol

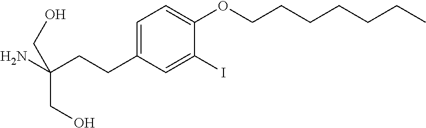

In analogy to the procedure described for the synthesis of example G, the title compound is prepared from 2-amino-2-[2-[4-(heptyloxy)phenyl]ethyl]-1,3-propanediol.

LCMS Rt_{B}=1.20 min; [M]⁺=436.0

¹H-NMR (400 MHz, DMSO-d₆) δ ppm 7.60 (s, 1H); 7.12-7.18 (m, 1H); 6.8-6.78 (m, 1H); 4.42 (br s, 2H); 3.92 (m, 2H); 3.09-3.25 (m, 4H); 2.6-2.67 (m, 2H); 1.62-1.73 (m, 2H); 1.38-1.56 (m, 4H); 1.2-1.36 (m, 6H); 0.81-0.88 (m, 3H).

### Example J

### Phosphoric acid mono[(S)-2-amino-2-hydroxymethyl-3-iodo-4-octylphenyl)butyl]ester

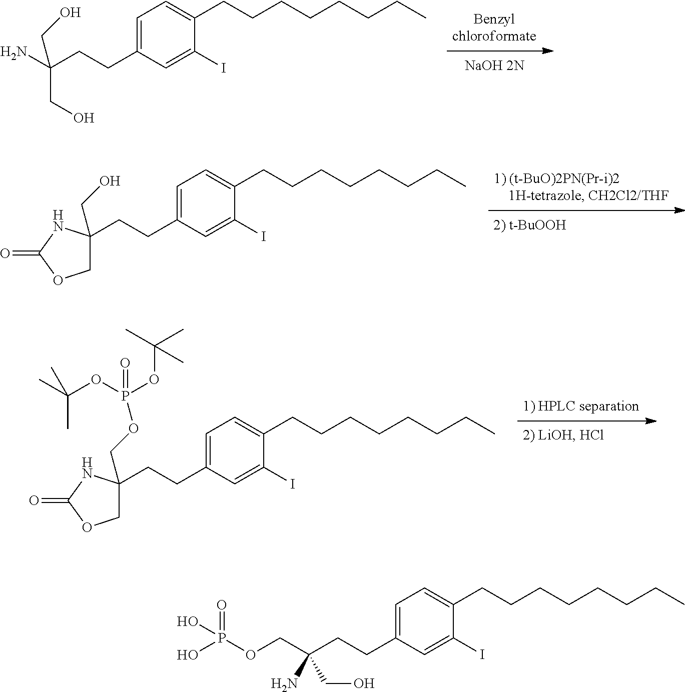 [0274] a) 4-hydroxymethyl-4-[2-(3-iodo-4-octylphenyl)ethyl]oxazolidin-2-one

Benzyl chloroformate (0.37 mL, 2.47 mmol) is added to a suspension of the compound described in Example 7 (1 g, 2.3 mmol) in 2N NaOH (10 mL). The mixture is kept at room temperature overnight. Then, the mixture is acidified with 1N HCl and extracted with methylene chloride. The organic phase is dried over Na₂SO₄, filtered and concentrated. The residue is purified on a silica gel column to give the title compound as a white powder.

UPLCMS Rt_{D}=3.36 min; [M+H]⁺=460

¹H-NMR (400 MHz, DMSO-d₆) δ ppm 7.65 (s, 1H); 7.59 (d, 1H); 7.04-7.23 (m, 2H); 5.08 (t, 1H); 4.16 (d, 1H); 4.06 (d, 1H); 3.30-3.41 (m, 2H); 2.55-2.69 (m, 2H); 2.42-2.47 (m, 2 H); 1.63 (dd, 2H); 1.47-1.52 (m., 2H); 1.18-1.25 (m, 10H); 0.74-0.91 (m, 3H)
b) Phosphoric acid mono-{(R/S)-4-[2-(3-iodo-4-octylphenyl)ethyl]oxazolidin-2-one}ester

To a solution of 4-hydroxymethyl-4-[2-(3-iodo-4-octylphenyl)ethyl]oxazolidin-2-one (815 mg, 1.77 mmol) in dichloromethane (5 mL) and THF (5 mL) at 0° C. is added 1H-tetrazole (621 mg, 8.87 mmol) and di-tert-butyl diethyl-phosphoramidite (1.59 mL, 5.32 mmol). After 18 hours at room temperature, hydrogen peroxide (0.54 mL, 17.7 mmol) [30% in water] is added drop wise and then the mixture is stirred at room temperature for an additional 90 minutes. The reaction mixture is quenched with saturated Na₂S₂O₃ and the water phase is extracted with dichloromethane. The organic layer is dried over Na₂SO₄, filtered and concentrated. The crude product is purified by flash chromatography on silica gel to give compound as a yellow oil.

UPLCMS Rt_{D}=4.56 min; [M]⁺=651

¹H-NMR (400 MHz, DMSO-d⁶) δ ppm 7.87 (s, 1H); 7.67 (s, 1H); 7.08-7.25 (m, 2H); 4.06-4.21 (m, 2H); 3.78 (d, 2H); 1.65-1.86 (m, 2H); 1.46-1.52 (m, 2H); 1.40 (s, 18H); 1.17-1.34 (m, 14H); 0.84 (t, 3H)
c) Separation of enantiomers of phosphoric acid mono-{(R/S)-4-[2-(3-iodo-4-octylphenyl)ethyl]oxazolidin-2-one}ester is performed by HPLC on Chiralpak AS-PREP column at preparative scale (heptane/iEtOH/MeOH 80/10/10 as mobile phase).
d) Phosphoric acid mono-{(S)-2-amino-2-hydroxymethyl-4-[2-(3-iodo-4-octylphenyl)butyl]}ester

To a solution of phosphoric acid mono-{(S)-4-[2-(3-iodo-4-octylphenyl)ethyl]oxazolidin-2-one}ester (30 mg, 0.046 mmol) in ethanol (0.5 mL) is added lithium hydroxide (0.5 mL, 2.09 mmol) 10% solution. After 20 hours at 60° C., the reaction mixture is cooled to room temperature and stirred for 2 days. Concentrated HCl (0.5 mL) is added and the solution is stirred at room temperature for 1 hour. The mixture is neutralized with NaOH 4N and concentrated. The residue is taken up in dichloromethane (2 mL), and filtered on Hyflo and the filter cake is washed twice with dichloromethane. The solution is concentrated to give the title compound as a white powder.

LCMS Rt_{B}=1.41 min; [M]⁺=514

### Example K

### Phosphoric acid mono-{(S)-2-amino-2-hydroxymethyl-4-[2-(2-iodo-4-octylphenyl)butyl]}ester

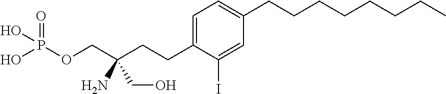

In analogy to the procedure described for the synthesis of example J, the title compound is prepared from 2-amino-2-[2-(2-iodo-4-octylphenyl)ethyl]-1,3-propandiol.

LCMS Rt_{B}=1.32 min; [M]⁺=513.8

### Example L

### Phosphoric acid mono-{(S)-2-amino-2-hydroxymethyl-4-[2-(3-iodo-4-(heptyloxy)phenyl)butyl]}ester

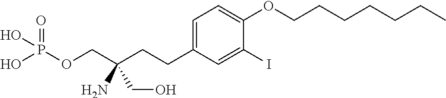

In analogy to the procedure described for the synthesis of example J, the title compound is prepared from 2-amino-2-[2-[3-iodo-4-(heptyloxy)phenyl]ethyl]-1,3-propanediol LCMS Rt_{B}=1.28 min; [M+H]⁺=516.0

### Example M

### Preparation of Aryl Neopentyl Boronate Precursor

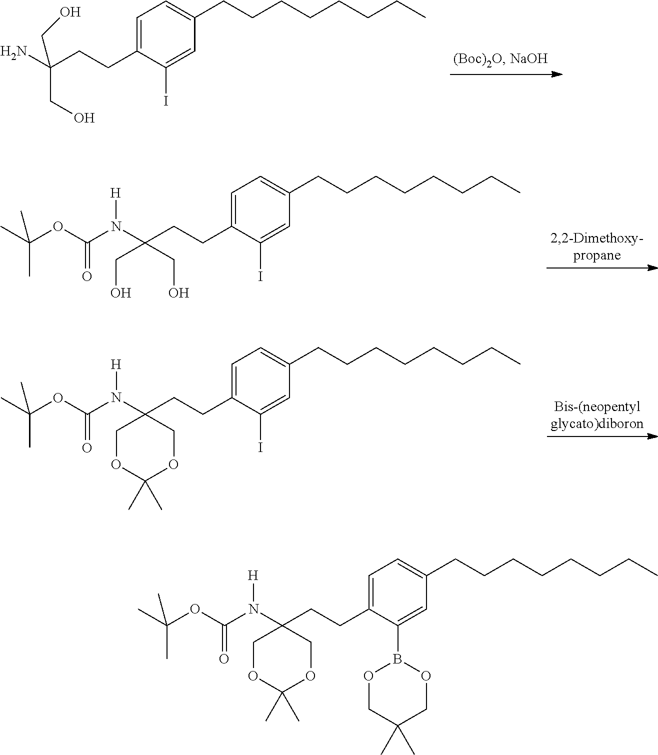 [0292] a) [1,1-Bis-hydroxymethyl-3-(2-iodo-4-octyl-phenyl)-propyl]-carbamic acid tert-butyl ester

A mixture of 2-amino-2-[2-(2-iodo-4-octylphenyl)ethyl]-1,3-propandiol (110 mg, 0.25 mmol), (Boc)₂O (0.09 mL, 0.38 mmol) and NaOH 1M (0.28 mL, 0.28 mmol) in dioxane (5 mL) is stirred at room temperature overnight. The reaction mixture is extracted with ethyl acetate and the organic layer is dried over sodium sulfate, filtered and concentrated. The crude product is purified by flash chromatography on silica gel to give title compound as a colorless oil.

LCMS Rt_{B}=1.99 min; [M]⁺=533.8

1H-NMR (360 MHz, CDCl₃) δ ppm 7.67 (s, 1H); 7.07-7.15 (m, 2H); 5.06 (s, 1H); 3.90 (dd, 2H); 3.67 (dd, 2H); 3.42 (bs, 2H); 2.63-2.74 (m, 2H); 2.49-2.63 (m, 2H); 1.81-1.96 (m, 2H); 1.56-1.60 (m, 2H); 1.49 (s, 9H); 1.30-1.43 (m, 10H); 0.87-0.96 (m, 3H)
b) {5-[2-(2-Iodo-4-octyl-phenyl)-ethyl]-2,2-dimethyl-[1,3]dioxan-5-yl}-carbamic acid tert-butyl ester

To a solution of [1,1-Bis-hydroxymethyl-3-(2-iodo-4-octyl-phenyl)-propyl]-carbamic acid tert-butyl ester (230 mg, 0.43 mmol) in DMF (2 mL) is added 2,2-dimethoxy-propane (5.3 mL, 43.1 mmol), acetone (3.2 mL, 43.1 mmol) and pTsOH.H₂O (8.2 mg, 0.043 mmol) at RT. Then, the reaction mixture is stirred for 1 hour. The solution is quenched with a satured solution of NaHCO₃, extracted with ethyl acetate then the organic layer is dried over Na₂SO₄, filtered and concentrated. The crude product is purified by flash chromatography on silica gel to give 240 mg of title compound as a colorless oil.

LCMS Rt_{B}=1.92 min; [M+H]⁺=574.2

1H NMR (360 MHz, CDCl₃) δ ppm 7.55 (s, 1H) 6.93-7.08 (m, 2H) 4.90 (br. s., 1H) 3.82 (d, 2H) 3.60 (d, 2H) 2.49-2.64 (m, 2H) 2.30-2.47 (m, 2H) 1.77-1.94 (m, 2H) 1.44-1.46 (m, 2H) 1.37-1.43 (m, 9H) 1.36 (s, 3H) 1.34 (s, 3H) 1.16-1.30 (m, 10H) 0.66-0.92 (m, 3H).
c) (2,2-Dimethyl-5-{2-[4-octyl-2-(4,4,5,5-tetramethyl-[1,3,2]dioxaborolan-2-yl)-phenyl]-ethyl}-[1,3]dioxan-5-yl)-carbamic acid tert-butyl ester

A 2-necked, 25 mL round-bottomed flask is charged with PdCl₂(dppf) (14.2 mg, 0.017 mmol), KOAc (51.3 mg, 0.52 mmol) and bis-(neopentyl glycato)diboron (43.3 mg, 0.19 mmol) and flushed with nitrogen. A solution of {5-[2-(2-Iodo-4-octyl-phenyl)-ethyl]-2,2-dimethyl-[1,3]dioxan-5-yl}-carbamic acid tert-butyl ester (100 mg, 0.17 mmol) in DMSO (1 mL) is added and the solution is stirred for 3 h, at 50° C. The product is extracted into ethyl acetate, washed with water, and dried over anhydrous sodium sulfate. The organic solvent is removed under reduced pressure and the product is purified by flash chromatography on silica gel to give the title compound as a white solid.

LCMS Rt_{B}=2.18 min; [M+H]⁺=560.2.

### Example N

### Preparation of pinacol boronate precursor: (2,2-Dimethyl-5-{2-[4-octyl-2-(4,4,5,5-tetramethyl-[1,3,2]dioxaborolan-2-yl)-phenyl]-ethyl}-[1,3]dioxan-5-yl)-carbamic acid tert-butyl ester

A 20 mL Supelco-vial is charged with 1,1′-bis(diphenylphosphino)ferrocene-palladium(II)dichloride dichloromethane complex (8.54 mg, 10.46 μmol), potassium acetate (103 mg, 1.046 mmol) and bis(pinacolato)diboron (97 mg, 0.38 mmol) and flushed with argon. A solution of {5-[2-(2-Iodo-4-octyl-phenyl)-ethyl]-2,2-dimethyl-[1,3]dioxan-5-yl}-carbamic acid tert-butyl ester (200 mg, 0.349 mmol) in DMSO (6 mL) is added and the solution is stirred for 4 hours at 80° C. The reaction mixture is quenched with H₂O and extracted with ethyl acetate. The organic layer is washed with brine and dried over sodium sulfate, filtered, concentrated under reduced pressure and the crude product is purified by flash chromatography on silica gel to give the title compound as a white powder.

LCMS Rt_{D}=1.90 min; [M+H]⁺=574.4

¹H NMR (360 MHz, DMSO-d₆) δ ppm 7.44-7.46 (m, 1H); 7.21 (d, 1H); 7.00 (d, 1H); 6.51 (br. s., 1H); 3.92 (d, 2H); 3.69 (d, 2H) 2.64-2.72 (m, 2H); 2.52-2.56 (m, 2H); 1.78-1.85 (m, 2H); 1.49-1.58 (m., 2H); 1.41-1.46 (m, 9H); 1.34-1.36 (m, 6H); 1.30-1.32 (m, 12H); 1.25-1.29 (m, 10H); 0.84-0.90 (m, 3H).

### Example O

### Preparation of trifluoroboronate precursor: (2,2-Dimethyl-5-{2-[4-octyl-2-(trifluoroborolan-2-yl)-phenyl]-ethyl}-[1,3]dioxan-5-yl)-carbamic acid tert-butyl ester

To a solution of pinacolylboronate (200 mg, 0.35 mmol) in methanol (2 mL) is added aqueous potassium hydrogen fluoride (0.45 mL, 1.97 mmol). The resulting slurry is stirred at RT for 15 min, concentrated in vacuo and then dissolved in hot acetone, filtered and concentrated in vacuo. The filtrate is recrystallized from hot methanol to afford the title compound as a white solid.

LCMS Rt_{D}=1.75 min; [M-K]⁺=514.3

¹H NMR (360 MHz, DMSO-d₆) δ ppm 7.16 (s, 1H); 6.75 (s, 2H); 6.39 (br. s., 1H); 3.98 (d, 2H); 3.59 (d, 2H); 2.52-2.56 (m, 2H); 2.39-2.46 (m, 2H); 1.75-1.82 (m, 2H); 1.48-1.56 (m, 2H); 1.43 (s, 9H); 1.31-1.34 (m, 6H); 1.23-1.30 (m, 10H); 0.82-0.92 (t, 3H)

### Example P

### General Procedure for the Preparation of Labeled Product with ¹²³I Via Boronate Precursors

No-carrier-added Na¹²³I (74 MBq in 0.1% aqueous NaOH) is placed into a 2 mL Wheaton vial containing the arylboronate precursor (100 mL of 4 10⁻² M solution in 50% aqueous THF). The reaction vial is sealed, covered with aluminum foil, and the mixture stirred for 5 min at room temperature. A drop of 10% aqueous sodium thiosulfate is added to decompose the excess of iodine. The ¹²³I-intermediate is deprotected in the presence of 3N HCl in ethyl acetate to give the desired ¹²³I-compound. The radioiodinated product is isolated by passing it through a silica gel Sep-pak cartridge using pentane:EtOAc (50:1) as eluent.

GTPγS Binding Assay Using S1P Receptor/CHO Membranes Preparation

The assay is based on the SPA technology (Amersham) and run in a 96 well format. Membranes are prepared from CHO cells stably expressing the S1P receptor of interest. Aliquots are stored at −80° C. Membranes (5-10 μg/well) resuspended in assay buffer (20 mM HEPES, pH7.4, 100 mM NaCl, 10 mM MgCl₂ and 0.1% fat free BSA) containing 25 μg/mL Saponin and 10 μM GDP are mixed WGA-coated SPA beads (final conc. 1 mg/well). Ligand and [³⁵S]GTPγS (1250 Ci/mmol, final concentration 0.2 nM) are added and the plate sealed. After incubation at room temperature for 120 minutes under constant shaking the plates are centrifuged for 10 minutes at 1000×g to pellet the SPA beads. Then the plates are measured in a TopCount NXT instrument (Packard) and the data analyzed using GraphPad PRISM software.

In particular the EC₅₀ values in nM for the following compounds at various S1P receptors are shown in the table below:

| | S1P-1 | S1P-3 | S1P-4 | S1P-5 |
|---|---|---|---|---|
| Example B | 0.69 | 1.2 | 0.84 | 0.62 |
| Example D | 1.3 | 3.0 | 1.9 | 1.1 |
| Example F | 1.6 | 8.6 | 2.9 | 1.4 |
| Example J | 0.17 | 84 | 4.9 | 2.0 |
| Example K | 0.24 | 46 | 7.1 | 1.5 |
| Example L | 0.3 | — | — | 0.7 |

Percent of Lymphocyte Depletion in Lewis Rats

The lymphocyte homing property may be measured in following Blood Lymphocyte Depletion assay:

A S1P receptor agonist or the vehicle is administered intravenously to rats. Tail blood for hematological monitoring is obtained on day-1 to give the baseline individual values, and at 2, 4, 8, 24, and 48 hours after application. In this assay, the S1P receptor agonist depletes peripheral blood lymphocytes, e.g. by 50%, when administered at a dose of e.g. <20 mg/kg. Preferred S1P receptor agonists are further compounds which in addition to their S1P binding properties internalize/desensitize S1P receptors, thereby antagonizing inflammatory processes driven by lysophospholipids, including i.e. sphingosine 1-phosphate (S1P), sphingophosphorylcholine (SPC), lysophosphatidic acid (LPA), and others, on vasculature cells, e.g. endothelial cells. The internalization/desensitization capacity of compounds will be determined using CHO cells transfected with a human myc-tagged S1P receptor.

| | Time post treatment (hours) | | |
|---|---|---|---|
| | 2 | 10 | 24 |
| FTY720 | 71 | 86 | 86 |
| Example G | 51 | 88 | 87 |
| Example E | 64 | 82 | 80 |
| Example H | 67 | 89 | 89 |

The radiolabelled FTY720 derivatives of the invention may be used, for instance, to determine their distribution and concentration ex vivo in rats, or in vivo in non-human primates and man, by using methods known to the skilled person, for example as described by Pauwels et al. (Current Pharmaceutical Design 2009, 15, 928-934) or Bergstroem et al (Eur J Nucl Med 1997, 24, 596-601).

Variations, modification, and other implementations of what is described herein will occur to those of ordinary skill in the art without departing from the spirit and the essential characteristics of the present invention. Accordingly the scope of the invention is to be defined not by the preceding illustrative description and examples but instead by the following claims, and all changes that come within the meaning and range of equivalency of the claims are intended to be embraced therein.

1. A compound, or pharmaceutically acceptable salt thereof, wherein said compound is selected from
a compound of formula I 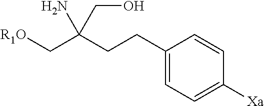
wherein
Xₐ is C₁₋₁₀ alkyl or OC₁₋₉ alkyl;
R₁ is H or C₁₋₆ alkyl, or PO₃H₂;
and wherein at least one hydrogen atom is replaced by a iodine or bromine atom;
a compound of formula Ia, 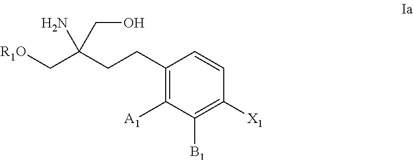
wherein
R₁ is as defined above;
at least one of A₁ and B₁ is iodine or bromine, the other is hydrogen; and
X₁ is C₁₋₁₀ alkyl or OC₁₋₉ alkyl;
a compound of formula Ib 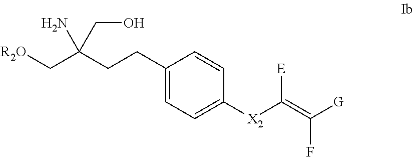
wherein
R₂ is H, C₁₋₆ alkyl, or PO₃H₂;
at least one of E, F and G is iodine or bromine and the others are hydrogen; and
X₂ is C₁₋₈ alkyl or OC₁₋₇ alkyl.

2. A compound or pharmaceutically acceptable salt according to claim 1, wherein the compound is selected from

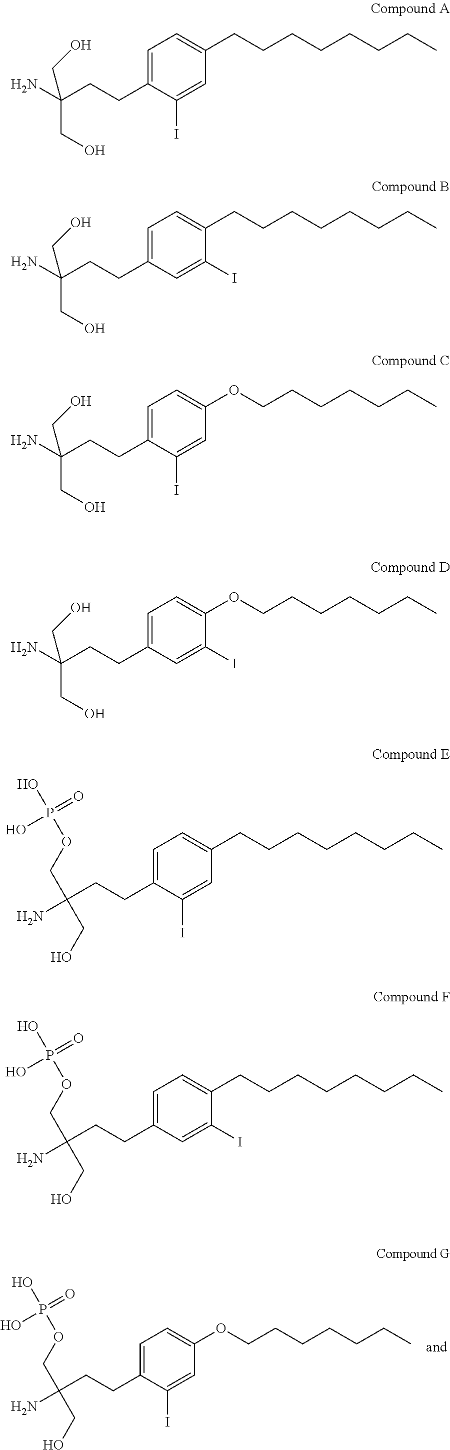

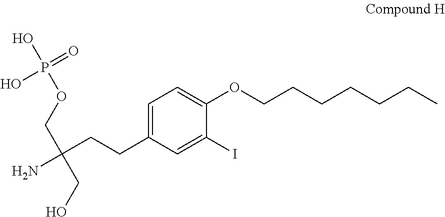

3. A compound or pharmaceutically acceptable salt according to claim 1, wherein the compound is selected from

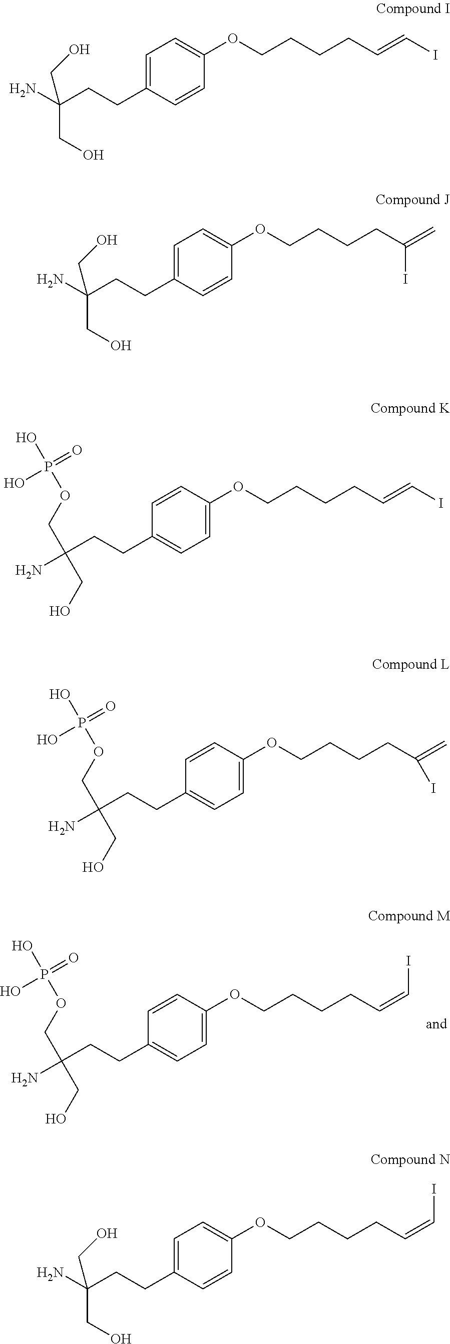

4. A radiolabelled compound or pharmaceutically acceptable salt according to claim 1, comprising at least one atom selected from the group consisting of ¹²³I, ¹²⁵I, ¹²⁴I, ¹³¹I, ⁷⁵Br and ⁷⁶Br.

5. A radiolabelled compound or pharmaceutically acceptable salt according to claim 2, wherein the compound is selected from Compound A, C, E and G, and wherein the compound contains an iodine atom which is ¹²³I or ¹²⁴I.

6. A method for diagnosing a disease or disorder in which S1P receptor expression is altered, wherein said disease or disorder is selected from inflammatory diseases, autoimmune diseases, demyelinating diseases, and brain diseases, in a patient in need thereof, comprising:
a) administering to said patient a radiolabelled compound or pharmaceutically acceptable salt according to claim 1; and
b) detecting or measuring the radiation emitted from said compound or salt in vivo using an appropriate imaging instrument.

7. A method according to claim 6 wherein the disease is multiple sclerosis.

8-11. (canceled)

12. A method of predicting if a patient suffering from a disease or disorder selected from inflammatory diseases, autoimmune diseases, demyelinating diseases and brain diseases, will have a favorable response to pharmacotherapy with a drug that is a S1P receptor modulator, comprising:
a) administering to the patient an effective amount of a radiolabelled compound or pharmaceutically acceptable salt according to claim 1, and
b) detecting or measuring the radiation emitted from said compound or salt with an appropriate imaging instrument.

13. A method for monitoring the effectiveness in a patient of a pharmacotherapy for a disease or disorder wherein S1P receptor expression is altered, wherein said method comprises:
a) administering to the patient an effective amount of a radiolabelled compound or pharmaceutically acceptable salt according to claim 1, and
b) detecting or measuring the radiation emitted in vivo from said compound or salt with an appropriate imaging instrument.

14. A method according to claim 6, wherein the compound or pharmaceutically acceptable salt that is administered is. 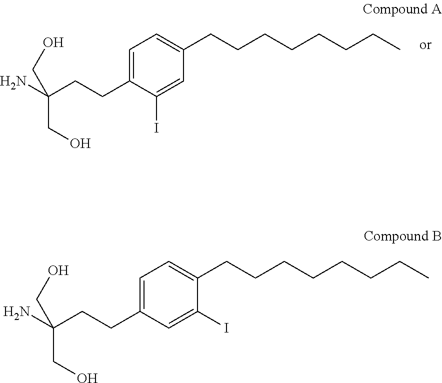
or
wherein the iodine atom depicted in Compound A and Compound B is ¹²³I, ¹²⁵I, ¹²⁴I, or ¹³¹I, or a phamaceutically acceptable salt thereof.

15. (canceled)

16. A method according to claim 12, wherein the compound or pharmaceutically acceptable salt that is administered is 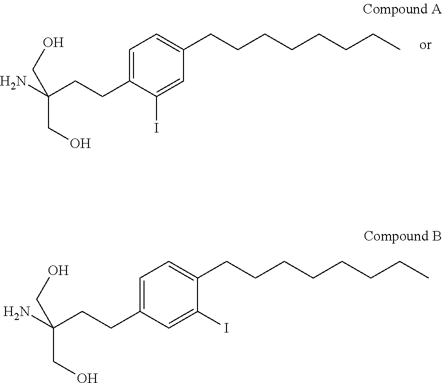
wherein the iodine atom depicted in Compound A and Compound B is ¹²³I, ¹²⁵I, ¹²⁴I, or ¹³¹I, or a phamaceutically acceptable salt thereof.

17. A method according to claim 13 wherein the compound or pharmaceutically acceptable salt that is administered is 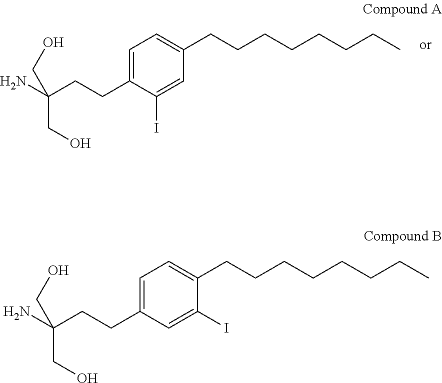
or
wherein the iodine atom depicted in Compound A and Compound B is ¹²³I, ¹²⁵I, ¹²⁴I, or ¹³¹I, or a phamaceutically acceptable salt thereof.

18. A method according to claim 6, wherein the imaging instrument is a positron emission tomography imaging instrument or a single photon emission computed tomography imaging instrument.

19. A method according to claim 6, wherein the compound or pharmaceutically acceptable salt that is administered is a radiolabelled derivative of FTY720.

20. A method according to claim 12, wherein the compound or pharmaceutically acceptable salt that is administered is a radiolabelled derivative of FTY720.

21. A method according to claim 13, wherein the compound or pharmaceutically acceptable salt that is administered is a radiolabelled derivative of FTY720.

22. A method according to claim 6, wherein the compound or pharmaceutically acceptable salt that is administered is a radiolabelled compound according to claim 3.

23. A method according to claim 12, wherein the compound or pharmaceutically acceptable salt that is administered is a radiolabelled compound according to claim 3.

24. A method according to claim 13, wherein the compound or pharmaceutically acceptable salt that is administered is a radiolabelled compound according to claim 3.

25. A method according to claim 6, wherein the compound or pharmaceutically acceptable salt that is administered is a radiolabelled compound according to claim 2.

26. A method according to claim 12, wherein the compound or pharmaceutically acceptable salt that is administered is a radiolabelled compound according to claim 2.

27. A method according to claim 13, wherein the compound or pharmaceutically acceptable salt that is administered is a radiolabelled compound according to claim 2.

28. A method according to claim 6, wherein the compound or pharmaceutically acceptable salt that is administered is a radiolabelled compound according to claim 4.

29. A method according to claim 12, wherein the compound or pharmaceutically acceptable salt that is administered is a radiolabelled compound according to claim 4.

30. A method according to claim 13, wherein the compound or pharmaceutically acceptable salt that is administered is a radiolabelled compound according to claim 4.
